(19) United States
(12) Patent Application Publication
(10) Pub. No.: US 2021/0223254 A1
(43) Pub. Date: Jul. 22, 2021

(54) Methods for Multicolor Multiplex Imaging

(71) Applicant: Ultivue, Inc., Cambridge, MA (US)

(72) Inventors: Douglas Wood, Hingham, MA (US); Mark Bobrow, Lexington, MA (US); Mael Manesse, Medford, MA (US); Eloise Wheeler, Beverly, MA (US); Heike Boisvert, Somerville, MA (US)

(73) Assignee: Ultivue, Inc., Cambridge, MA (US)

(21) Appl. No.: 17/257,968

(22) PCT Filed: Jul. 2, 2019
(86) PCT No.: PCT/US19/40235

§ 371 Date: Jan. 5, 2021

Related U.S. Application Data
(60) Provisional application No. 62/695,381, filed on Jul. 9, 2018.

(51) Int. Cl. G01N 33/58; G01N 1/30; G06K 9/00; G06K 9/46

(52) U.S. Cl. CPC G01N 33/582; G01N 1/30; G06K 9/00134; G06K 2209/05; G06K 9/0014; G06K 9/4652; G01N 2458/10; G06K 9/4661

(57) ABSTRACT

Methods for multicolor multiplex imaging are provided herein in order to increase the number of targets that may be imaged given the number of fluorophores available or desired for use.

### CROSS-REFERENCE TO RELATED APPLICATIONS

This application claims the benefit of priority of Untied States Provisional Application No. 62/695,381, filed Jul. 9, 2018, the content of which are incorporated by reference herein in its entirety for any purpose.

### FIELD

This application relates generally to the field of detection and quantification of analytes (e.g., targets).

### BACKGROUND

Fluorescence microscopy is a powerful tool for detecting molecules in, for example, a biological system. For example, when imaging cells in tissue sections for pathology or in cell suspensions for cytology using a fluorescent microscope, it can be useful to collect signals from as many targets (markers) as possible from each slide. For this purpose, fluorescent labels are selected or designed to produce as narrow a band of emission as possible in order to minimize cross-talk between targets when multiple labels are imaged in an array of closely spaced spectral channels (i.e., spectral multiplexing). Without taking any additional steps, the number of targets that can be imaged is limited by the number of spectral channels in the imaging system (typically 6 or 7).

There is still a need for improved methods that allow multiplexing to expand the number of targets that can be interrogated using a limited number of fluorophores while achieving higher level of detection and quantification of the targets.

### SUMMARY

In accordance with the description, methods for testing for the presence of a plurality of targets are provided herein, in particular for multiplexed imaging when the number of targets that can be interrogated exceeds the number of labels available.

In one embodiment, a multicolor multiplex imaging method comprises:
(1) contacting a sample being tested for the presence of one or more targets with one or more target-specific binding partners, wherein each target-specific binding partner is linked to a nucleic acid strand and wherein target-specific binding partners of different specificity are linked to different nucleic acid strands;
(2) optionally removing unbound target-specific binding partners;
(3) contacting the sample with labeled imager strands, wherein in at least one occurrence of this step the labeled imager strands comprise (i) multiple labeled imager strands capable of binding the same nucleic acid strand associated with the target-specific binding partner, and to either the same or different domains within the nucleic acid strand, wherein the multiple imager strands comprise a different type of label and/or (ii) at least one labeled imager strand capable of binding the same nucleic acid strand associated with the target-specific binding partner, wherein the imager strand comprises more than one type of label;
(4) optionally removing unbound labeled imager strands;
(5) imaging the sample to detect bound labeled imager strands;
(6) optionally removing the bound labeled imager strands from the nucleic acid strands; and
(7) optionally repeating steps (1)-(6), or any subset thereof wherein imaging the sample to detect the bound labeled imager strands includes detecting Nₘ targets with N_{ch} labels used in the method wherein Nₘ is larger than N_{ch}.

In some embodiments, at least one of the labeled imager strand(s) of step (3)(i) and/or (ii) are capable of binding the same nucleic acid strand associated with the target-specific binding partner directly. In other embodiments, at least one of the labeled imager strand(s) of step (3)(i) and/or (ii) are capable of binding the same nucleic acid strand associated with the target-specific binding partner indirectly.

In some embodiments, labeled imager strands of step (3)(i) and/or (ii) capable of binding the same nucleic acid strand associated with the target-specific binding partner bind to the same domain within the nucleic acid strand. In other embodiments, labeled imager strands of step (3)(i) and/or (ii) capable of binding the same nucleic acid strand associated with the target-specific binding partner bind to a different domain within the nucleic acid strand.

In some embodiments, the method amplifies at least one signal. In other embodiments, the method does not amplify at least one signal. In some embodiments, the method results in at least one target being labeled with at least two different types of labels.

In another embodiment, a kit is provided for detecting Nₘ targets with N_{ch} labels provided in the kit wherein Nₘ is larger than N_{ch}, and the kit may comprise: 1) target-specific binding partners linked to nucleic acid strands, wherein target-specific binding partners of different specificity are linked to different nucleic acid strands; 2) labeled imager strands comprising (i) multiple labeled imager strands capable of binding the same nucleic acid strand associated with the target-specific binding partner, to either the same or different domains within the nucleic acid strand, wherein the multiple imager strands comprise a different type of label and/or (ii) at least one labeled imager strand capable of binding the same nucleic acid strand associated with the target-specific binding partner, wherein the imager strand comprises more than one type of label; and 3) optional buffers, amplification reagents, and/or reagents to remove bound imager strands.

In another embodiment, a system for detecting a plurality of targets from fluorescence spectral data, wherein the number of targets being detected, Nₘ, given the number of labels, N_{ch}, is represented by the following formula:

N_{ch}<Nₘ≤N_{ch}*(N_{ch}+1)/2,

where Nₘ and N_{ch} are an integer is provided. The system may comprise a fluorescent microscope, a light source, a detector, a computer processor operably connected with the detector; and a tangible non-transitory storage medium having computer-readable instructions embedded therein which, when loaded onto the computer processor, cause the processor to conduct the following:
(1) determining relative intensities in each spectral channel of a label or labels for each target;
(2) based on the determined relative intensities, grouping the targets into N_{ch}, mutually exclusive basis sets, the targets being the members of each basis set commonly having non-zero intensity in one of N_{ch} channels;
(3) given the relative spectral intensity of the members of each basis set, and given the measured intensity of the sample in each pixel in each channel, adjusting the levels of member of each basis set to produce the least error in matching the basis set to the measured intensities; and
(4) selecting the basis set with the least error and assigning each element of an output array with Nₘ values to the levels determined for the members of the selected set or to zero for members not of the selected set.

Steps (3)-(4) may be repeated for a portion or all of pixels of the input image. Steps (3)-(4) may be conducted in parallel for multiple pixels of the input image.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F show model images for simulation of imaging 6 targets with 3 labels in Example 1. In the model image of targets A-C (FIGS. 1A-1C, top row), the targets are shown in the lower left quadrant; in the model image of targets D and E (FIGS. 1D and 1E), the upper left quadrant; and in the model image of target F (FIG. 1E), the upper right quadrant.

FIGS. 2A-2C show model images encoded in three spectral channels (corresponding to three labels 1-3) in Example 1, respectively.

FIGS. 3A-3F show decoded images of 6 targets A-F in Example 1. Compare decoded images of targets A-C (FIGS. 3A-3C, top row) to model images in FIGS. 1A-1C, respectively; compare decoded images of target D-F (FIGS. 3D-3F, bottom row) to model images in FIGS. 1D-1F, respectively.

FIG. 4 shows the image of 6 targets decoded from 4 spectral channels (corresponding to labels 1-4) overlaid with the DAPI image in Example 2. Targets are shown as the following color: PDL1 shown in Red, PD1 in Green, Ki67 in magenta, DAPI in Blue, CK in Yellow, CD8 in turquoise, and CD3 in brown.

FIGS. 5A and 5B show comparison of the decoded image of targets PDL1, PD1, Ki67, and CK from the full decoding of 6 targets in Example 2 (FIG. 5A) with a consecutive section stained with the targets alone (no CD3 or CD8 staining) (FIG. 5B). PDL1 shown in Red, PD1 in Green, Ki67 in magenta, DAPI in Blue, and CK in Yellow.

FIGS. 6A and 6B show comparison of the decoded CD8 signal recovered from the full decoding of 6 targets in Example 2 (FIG. 6A) with a consecutive section with stained with the CD8 dual color reagent alone (average of the Cy5 and AF555 channels), overlaid with DAPI (FIG. 6B). CD8 is shown in red and DAPI in blue.

FIGS. 7A and 7B show comparison of the decoded CD3 signal recovered from the full decoding of 6 targets in Example 2 (FIG. 7A) with a consecutive section with stained with CD3 dual color reagent alone (average of the Cy5 and Cy7 channels), overlaid with DAPI (FIG. 7B). CD3 is shown in red and DAPI in blue.

FIGS. 8A-8E show a scheme of attaching Docking Strands to the target via a target-recognizing moiety. Specifically, FIG. 8A shows Attachment of the Docking Strand to the target-recognizing moiety without signal amplification. FIG. 8B shows attachment of the Docking Strand to the target-recognizing moiety with signal amplification using a branched structure, which can be created using processes such as HCR. FIG. 8C shows attachment of the Docking Strand to the target-recognizing moiety with signal amplification using a linear structure, which can be created using processes such as RCA. FIG. 8D shows modified hybridization chain reaction (HCR), where a docking site (domain b) is attached to one of the two hairpins of HCR, allowing introduction of multiple docking sites to one target-recognizing moiety. FIG. 8E shows rolling circle amplification to introduce multiple docking site (domain c-d) to one target-recognizing moiety. The following reference numbers are used in this figure. 101: Target. 102: Target-recognizing moiety. 103: Docking Strand. 120: Primer strand of the HCR reaction that is attached to the target-recognizing moiety. 121: one hairpin of HCR, which is attached with the docking site. 122: another hairpin of HCR. 123-126: Sequential hairpin assembly reactions. 127: Primer that is attached to the target-recognizing moiety. 128: linear template that can be circularized by ligation. 129: The ligation reaction. 130: Primer extension with DNA polymerase with strand-displacement activity. 131: multiple docking sites.

FIG. 9 shows sequential amplification, polymerization, and dendrimerization for amplified and removable signal.

FIG. 10 shows simultaneous amplification, polymerization, and dendrimerization for amplified and removable signal.

FIGS. 11A-11B show (FIG. 11A) sequential imaging with sequential amplification from HRP-like enzymes and (FIG. 11B) simultaneous imaging with sequential amplification from HRP-like enzymes.

FIGS. 12A-12D show removal of Imager Strand using nucleic acid-degrading enzymes. (FIG. 12A) General scheme. (FIG. 12B) Embodiments where there is a single deoxyuridine (dU) nucleotide in the Docking Strand-recognizing portion of the Imager Strand. (FIG. 12C) Embodiments where there are multiple dU nucleotides in the Docking Strand-recognizing portion of the Imager Strand. (FIG. 12D) Embodiments where the dU nucleotide is placed within the linkage between the Docking Strand-recognizing portion and the signal-generating moiety of the Imager Strand. 104: imager strand. 105: signal generating moiety of the imager strand. 106: linkage between the target-recognizing moiety and the docking strand. 107: optional linkage to additional docking strands. 120: primer strand of the hybridization chain reaction (HCR) that is attached. 201: dU as an example of a moiety that can be degraded enzymatically. 202: The enzymatic reaction to degrade dU. 203: The process where the remnant of the degradation reaction spontaneous dissociates from the Docking Strand.

FIGS. 13A-13F show removal of Imager Strand using polymerase enzymes. (FIG. 13A) A self-priming hairpin is placed at the 3′ end of the Imager Strand; the Imager Strand is removed using a polymerase with strand-displacement activity (e.g., phi29). (FIG. 13B) A self-priming hairpin is placed at the 3′ end of the Imager Strand which is linked to the signal-generating moiety via nucleic acid hybridization; the Imager Strand is removed using a polymerase with strand-displacement activity. (FIG. 13C) A self-priming hairpin is placed at the 3′ end of the Docking Strand; the Imager Strand is removed using a polymerase with strand-displacement activity. (FIG. 13D) A self-priming hairpin is placed at the 3′ end of the Imager Strand; the Imager Strand is removed using a polymerase with 5′-to-3′ exonuclease activity (e.g., DNA Polymerase I). (FIG. 13E) A self-priming hairpin is placed at the 3′ end of the Docking Strand; the Imager Strand is removed using a polymerase with 5′-to-3′ exonuclease activity. (FIG. 13F) The self-priming hairpin is replaced by a hybridized duplex with an extendable 3′ end. 301: Self-priming hairpin. 302: The reaction where the self-priming hairpin or the hybridized duplex with an extendable 3′ end is extended by the DNA polymerase with strand-displacement activity. 303: The short oligonucleotide that brings the signal-generating moiety to the Imager Strand via hybridization. 304: The reaction where the self-priming hairpin or the hybridized duplex with an extendable 3′ end is extended by the DNA polymerase with 5′-to-3′ exonuclease activity. 305: hybridized duplex with an extendable 3′ end. 306: Linkage between the target-recognizing moiety and the Docking Strand, wherein the linkage comprises covalent or non-covalent interactions.

FIGS. 14A-14D show various embodiments of exchange imaging, some using primer and intermediate strands in addition to imager and docking strands. FIG. 14A shows DNA-Exchange imaging with the use of an intermediate strand (401) to link an imager strand and a docking strand bound to a target through a target-recognition moiety. FIG. 14B illustrates a primer strand (404) used to amplify the number of docking strands associated with a target-binding complex, where the resulting amplified product (403) is attached to multiple docking sites (103) and can be imaged with an imager strand, directly or indirectly through an intermediate strand as shown. FIG. 14C shows amplification of the number of docking strands associated with a target using a primer strand to initiate a hybridization chain reaction and imaging with the addition of an imager strand, bound to docking strand through an intermediate strand. FIG. 14D shows amplification of the number of docking strands associated with a target using a primer strand as a template for ligation and rolling circle amplification, followed by the addition of an imager strand, bound to docking strands through intermediate strands for imaging.

### DESCRIPTION OF THE EMBODIMENTS

### I. Methods of Testing a Sample for the Presence of a Plurality of Targets

This application relates to methods and compositions for testing for the presence of a plurality of targets, in particular multiplex imaging when the number of targets that can be interrogated exceeds the number of labels available.

Targets/markers in tissue samples or in individual cells are often imaged using fluorescent probes that are selected or designed to produce as narrow a band of emission as possible in order to minimize cross-talk between targets when multiple labels are imaged in an array of closely spaced spectral channels. Without taking any additional steps, the maximum number of targets that can be imaged at a time is limited by the number of labels having emissions from respective spectral channel in the imaging system (typically N≤6 or 7).

The present application provides a method for detecting more than N targets from just N labels (or spectral channels). In accordance with the present disclosure, fluorescent labels may be provided such that they emit multiple, narrow emission peaks (i.e., a spectral bar code). Further, the relative intensity of the peaks is consistent throughout the imaging process, such that the measured intensity of the label in any one spectral channel can be used to predict its emission in selected other channels, including all other channels in use. Then, the targets to be labelled in the sample are organized into mutually exclusive groups such that, in any given pixel of the image, only the members of a single set of targets are expected to be present at a time. In accordance with the present disclosure, the method described here allows one to decode the signals of more than N targets with just N labels (or spectral channels). Accordingly, based on information encoded in the pattern of the multiple peaks of the labels, which set of labels are present in any each pixel can be determined.

In real biological samples, the targets of each basis set can be selected based on biological function of a given cell. For example, tumor cells, immune cells, or stroma cells often are spatially separated into different regions of pixels of the image of the sample.

In some embodiments in accordance with the present disclosure, when a user may have a limited number of labels (e.g., fluorophores) available and/or when a user may have more targets for interrogation than the number of available labels, a target-specific probe (or imager strand) may be provided with more than two labels. In some embodiment, a target-specific probe (or imager strand) may be provided with two labels (dual-label multiplexing). In some embodiment, a target-specific probe (or imager strand) may be provided with three labels.

DNA-based reagents used in DNA exchange imaging discussed below may provide probes for multi-label encoding with predictable emission in multiple spectral channels.

A. Multicolor Labeling

The term “multicolor labels” or “multicolor labeling” refers to labeling a target or targets with more than one type of label having emissions from different spectral channels. Different embodiments of multicolor labeling may be applied to different targets in multiplexed imaging.

In some embodiments, two different types of labels may be used to label a target or targets such that the labeled target(s) have emissions from two spectral channels in a multiplexing method (referred herein as dual-color (label) multiplexing). In other embodiments, more than two types of labels may be employed to label a target or targets such that the labeled target(s) have emissions from more than two spectral channels in a multiplexing method. In some embodiments, for some targets, two or more types of labels are used to label each target, and for other targets, a single type of label is used to label each target.

In some embodiments, a multicolor labeling multiplex imaging method comprises:
(1) contacting a sample being tested for the presence of one or more targets with one or more target-specific binding partners, wherein each target-specific binding partner is linked to a nucleic acid strand and wherein target-specific binding partners of different specificity are linked to different nucleic acid strands,
(2) optionally removing unbound target-specific binding partners,
(3) contacting the sample with labeled imager strands, wherein in at least one occurrence of this step the labeled imager strands comprise (i) multiple labeled imager strands capable of binding the same nucleic acid strand associated with the target-specific binding partner, and to either the same or different domains within the nucleic acid strand, wherein the multiple imager strands comprise a different type of label and/or (ii) at least one labeled imager strand capable of binding the same nucleic acid strand associated with the target-specific binding partner, wherein the imager strand comprises more than one type of label,
(4) optionally removing unbound labeled imager strands,
(5) imaging the sample to detect bound labeled imager strands,
(6) optionally removing the bound labeled imager strands from the nucleic acid strands, and
(7) optionally repeating steps (1)-(6), or any subset thereof wherein imaging the sample to detect the bound labeled imager strands includes detecting Nₘ targets with N_{ch} labels used in the method or kit wherein Nₘ is greater than N_{ch}.

In some embodiments, Nₘ is chosen from an integer of 3, 4, 5, 6, 7, 8, 9, 10, 11, 12, 13, 14, or 15, and N_{ch} is a smaller integer chosen from 2, 3, 4, 5, and 6. In some embodiments, the method results in at least one target being labeled with at least two different types of labels.

In some embodiments, at least one of the labeled imager strand(s) of step (3)(i) and/or (ii) are capable of binding the same nucleic acid strand associated with the target-specific binding partner directly. In other embodiments, at least one of the labeled imager strand(s) of step (3)(i) and/or (ii) are capable of binding the same nucleic acid strand associated with the target-specific binding partner indirectly.

In some embodiments, labeled imager strands of step (3)(i) and/or (ii) capable of binding the same nucleic acid strand associated with the target-specific binding partner bind to the same domain within the nucleic acid strand. In other embodiments, labeled imager strands of step (3)(i) and/or (ii) capable of binding the same nucleic acid strand associated with the target-specific binding partner bind to a different domain within the nucleic acid strand.

In some embodiments, the method amplifies at least one signal. In other embodiments, the method does not amplify at least one signal.

1. Relationship of Number of Colors and Number of Targets

In some embodiments, where multicolor multiplexing is employed, the number of targets being detected, Nₘ, given the number of labels, N_{ch}, (corresponding to N_{ch} spectral channels), is represented by the following formula (1):

N_{ch}<Nₘ≤N_{ch}*(N_{ch}+1)/2 (1)

where Nₘ and N_{ch} are an integer. For example, (i) when N_{ch}=2, Nₘ is greater than 2 and equal to or smaller than 4; (ii) when N_{ch}=3, Nₘ is greater than 3 and equal to or smaller than 7; (iii) when N_{ch}=4, Nₘ is greater than 4 and equal to or smaller than 12; and (iv) when N_{ch}=5, Nₘ is greater than 5 and equal to or smaller than 17.

In some embodiments, 3 targets are imaged using two different of fluorescent labels (Nₘ=3, N_{ch} 2). In some embodiments, 4, 5, or 6 targets are imaged using 3 different fluorescent labels (Nₘ=4, 5, or 6 when N_{ch} 3). In some embodiments, 5, 6, 7, 8, 9, or 10 targets are imaged using 4 different fluorescent labels (Nₘ=5, 6, 7, 8, 9 or 10, when N_{ch} 4). In some embodiments, 6, 7, 8, 9, 10, 11, 12, 13, 14, or 15 targets are imaged using 5 different fluorescent labels (Nₘ=6, 7, 8, 9, 10, 11, 12, 13, 14, or 15, when N_{ch}=5).

This multicolor multiplexing may be accomplished by any of the means explained herein.

a) Two-Channel Spectral Multiplexing

In some embodiments, the method provides imaging of three targets with two fluorescent labels having emission wavelength in two respective spectral channels. As shown in Table 1, Target A is double-color labeled with labels 1 and 2, and Targets B and C are single-color labeled with label 1 and label 2, respectively.

TABLE 1
Three targets imaged with two labels

| | Basis | Label 1 | Label 2 |
|---|---|---|---|
| Target | Set | (Ch. 1) | (Ch. 2) |
| A | 1 | X | X |
| B | 1 | X | |
| C | 2 | | X |

In this and subsequent tables, an X indicates that the label for a given target has an emission peak in a given channel (i.e., the labels for Target A has two emission peaks). The first two targets, A and B, are identified as belonging to one group, because they were designed to produce emission in Channel 1. Such a group of targets is referred herein as a “basis set” and the index number of a basis set indicates the channel number shared by all the members of the basis set. The third target, C, only produces emission in Channel 2, and is the only member of basis set 2 as A and B were arbitrarily batched together based on both having emissions in Channel 1. (In an alternative embodiment, the B and C could have been batched together and A dealt with individually.)

For the dual-color labeled target A, the relative brightness of the labels in the two spectral channels is measured. The relative brightness can be measured by collecting an image of a sample stained with Label 1 alone and comparing the brightness of the images in the two channels on a pixel-by-pixel basis. At any given pixel in the image, if emission is detected in Channel 1, this signal is produced by target A or B or a combination of both. The intensity of the emission in Channel 2, and the known relative ratio of the emission intensity from Target A in Channel 2 and in Chanel 1, are used to estimate the contribution of Target A to the intensity measured in Channel 1. When the predicted contribution is subtracted from the measured intensity in Channel 1, the remainder is the emission from Target B, the remaining member of basis set 1. The level of Marker C in this set is assumed to be zero.

When a pixel does not have significant emission in Channel 1, that pixel represents basis set 2, and the intensity in Channel 2 is used to measure the level of Target C since Target C is the only member of basis set 2. The levels of targets A and B in this case are assumed to be zero.

b) Three-Channel Spectral Multiplexing

In some embodiments, the method provides imaging 6 targets with three spectral channels (corresponding to three different labels) according to the following encoding rules shown in Table 2:

TABLE 2
Six targets imaged with three labels

| Target | Basis Set | Chan 1 | Chan 2 | Chan 3 |
|---|---|---|---|---|
| A | 1 | X | X | |
| B | 1 | X | | X |
| C | 1 | X | | |
| D | 2 | | X | X |
| E | 2 | | X | |
| F | 3 | | | X |

In this case, when a pixel has significant emission in channel 1, the emission measured in Channels 2 and 3, and the known relative intensity ratios of the emission for Targets A and B in those respective channels to the emission in Channel 1 are used to estimate their contribution to Channel 1. The level of Target C is determined from subtracting those estimated values from the measured intensity in Channel 1. The levels of targets D, E and F are assumed to be zero in Channel 1.

If a pixel does not have significant emission in Channel 1, then it is treated in the same way as done in the previous 2-channel multiplexing (note that the pattern of encoding for targets D, E and F is the same as targets A, B, and C in Table 1).

c) Four-Channel Spectral Multiplexing

In some embodiments, the method provides imaging 10 targets with four spectral channels according to the following encoding rules shown in Table 3:

TABLE 3
Ten targets imaged with four labels

| Target | Basis Set | Chan 1 | Chan 2 | Chan 3 | Chan 4 |
|---|---|---|---|---|---|
| A | 1 | X | X | | |
| B | 1 | X | | X | |
| C | 1 | X | | | X |
| D | 1 | X | | | |
| E | 2 | | X | X | |
| F | 2 | | X | | X |
| G | 2 | | X | | |
| H | 3 | | | X | X |
| I | 3 | | | X | |

In this case, when a pixel has significant emission in Channel 1, the emission measured in Channels 2-4, and the known relative intensity ratios of the emission for Targets A, B, and C in those respective channels to the emission in Channel 1 are used to estimate their contribution to Channel 1. The level of Target D is determined from subtracting that estimated values from the measured intensity in Channel 1. The levels of targets E-J are assumed to be zero in Chanel 1.

If a pixel does not have significant emission in Channel 1, then it is treated in the same way as done in the previous 3-channel multiplexing (note that the pattern of encoding for targets E-J is the same as targets A-E in Table 2).

By extension, in some embodiments, the number of targets being detected, Nₘ, given the number of labels, N_{ch}, (corresponding to N_{ch} spectral channels), is determined by the following formula (2):

Nₘ=N_{ch}*(N_{ch}+1)/2 (2)

It is not necessary that all of the potential combinations be present in the sample. In the encoding rules, therefore, rows of these tables can be eliminated without affecting the decoding process.

In order for the decoding to be successful, each pixel or resolution element should contain the emission from the target of only one basis set. Every pixel or resolution element of the system is independent, so an adjacent pixel, or any other pixel in the image, can contain emission from a different basis set.

B. Multicolor Labeling Reagents

Various probes with more than one label attached may be used for the imaging method according to the present disclosure. In some embodiments, fluorescent molecules with the desired multiple peaked spectra may be prepared by the methods of organic or inorganic chemistry.

In some embodiments, labeled imaging nucleic acid strands used for DNA exchange imaging may be used (e.g., as described in WO 2018/107054, the entire content of which is incorporated by reference).

1. Reagents Used in Exchange Imaging

Exchange imaging is a method to achieve high multiplexing capability so that many targets can be imaged on the same sample. The central concept of Exchange Imaging involves the following steps: (1) attaching different decodable information-carrying molecules (called docking strands) to different target-specific binding partners (such as but not limited to an antibody that recognizes a target), wherein target-specific binding partners of different specificity (i.e., binding different targets) are linked to different docking strands and optionally removing unbound target-specific binding partners (2) using a set of molecules (called imager strands), each specifically recognizing a docking strand and carrying an observable moiety, to label a subset of docking strands, and imaging the corresponding subset of targets, (3) extinguishing the signal from the bound labeled imager strand by removing the set of imager strands used in step 2, removing the observable moiety from the imager strand, or inactivating the observable moieties on such imager strands, and (4) using another set of imager strands, each specifically recognizing a docking strand and carrying an observable moiety, to label another subset of docking strands, and imaging the corresponding subset of targets, (5) optionally, steps 3 and 4 can be repeated to visualize multiple subsets of targets. End users will readily appreciate that not all steps should be repeated in all experiments. For example, in the last round of imaging, there would be no need to extinguish the signal from the bound labeled imager strand because no further imaging strands would be applied.

One non-limiting example of Exchange Imaging is DNA Exchange Immunofluorescence, where one uses antibodies as the target-recognizing molecules to image target proteins or other biomolecules, uses DNA oligonucleotides as docking strands, and uses DNA oligonucleotides that are complementary to the docking strands and labeled with at least one observable moiety (such as a fluorophore) as the imager strands. By bound labeled imager strand, we aim to distinguish the labeled imager strand that has, at one point, bound to the docking strand from the excess labeled imager strand that did not bind to a docking strand. During the process of extinguishing the signal, the so-called bound labeled imager strand may remain bound to the docking strand or it may not remain bound to the docking strand.

In some embodiments, imaging the sample to detect bound labeled imager strands detects the presence of bound labeled imager strands. In some embodiments, imaging the sample to detect bound labeled imager strands detects the presence, location, and/or number of bound labeled imager strands.

When two different colored labels are co-localized to the same pixel, it provides a distinctive signal as compared to any single label. In some embodiments, with labels 1 and 2, a user can interrogate three targets if the imager strand corresponding to target A has label 1, the imager strand binding corresponding to target B has label 2, and user provides two sets of imager strands corresponding to target C including some with label 1 and some with label 2. Target C is imaged in both the label 1 and label 2, whereas target A is imaged only with the label 1 and target B is imaged with label 2.

a) Multiple Imager Strands Having Different Labels

Different embodiments of multicolor labeling may be applied to different targets in multiplexed imaging.

In some embodiments, in at least one occurrence, labeled imager strands comprise multiple labeled imager strands capable of binding the same nucleic acid strand associated with the target-specific binding partner, to either the same or different domains within the nucleic acid strand, wherein the multiple imager strands comprise a different type of label.

In some embodiments, in at least one occurrence, labeled imager strands comprise multiple labeled imager strands capable of binding the same nucleic acid strand associated with the target-specific binding partner bind to the same domain within the nucleic acid strand. In other embodiments, labeled imager strands capable of binding the same nucleic acid strand associated with the target-specific binding partner bind to a different domain within the nucleic acid strand.

In some embodiments, the multiple labeled imager strands comprise a first imager strand with a first label attached and a second imager strand with a second label attached, and both the first and second imager strands have a nucleotide sequence complementary to a same domain of the docking strand.

For example, when the plurality of imager strands, each linked with one of two different types of labels are introduced together in solution to the sample, which has been stained as before with an antibody linked with the corresponding docking strand, the complementary strands with the two labels compete for binding sites in a random fashion. As a result, the sample has some fraction of the desired antibody bound to one color type of label and the corresponding fraction bound to the other color type of label. Because there are many binding sites in a resolution element (pixel), the pixel appears to have two colors for that antibody. One advantage of preparing multiple imager strands linked with different labels is that by adjusting the relative concentrations of the different labeled imager strands in solution, we can adjust the relative brightness of each in the spectral channels. This is desirable because the decoding process (see below) works best with the signals that are being decoded are not grossly different in magnitude from each other.

In some embodiments, the multiple labeled imager strands capable of binding the same nucleic acid strand associated with the target-specific binding partner comprise an identical nucleotide sequence.

b) At Least One Labeled Imager Strand Comprising More than One Label

In some embodiment, in at least one occurrence, labeled imager strands comprise at least one labeled imager strand comprising more than one label. For example, a single imager strand may have two colors of fluorophores attached to it, or even more colors of fluorophores attached to it.

In some embodiments, in at least one occurrence, labeled imager strands comprise multiple labeled imager strands capable of binding the same nucleic acid strand associated with the target-specific binding partner (wherein at least one labeled imager strand comprises more than one label), to either the same or different domains within the nucleic acid strand, wherein the multiple imager strands comprise a different type of label (or pattern of labels).

In some embodiments, in at least one occurrence, the at least one labeled imager strand comprising more than one label binds to the same domain within the nucleic acid strand. In other embodiments, the at least one labeled imager strand comprising more than one label binds to a different domain within the nucleic acid strand.

c) Amplification Facilitates Multicolor Labeling

In some embodiments, the method amplifies at least one signal. In other embodiments, the method does not amplify at least one signal.

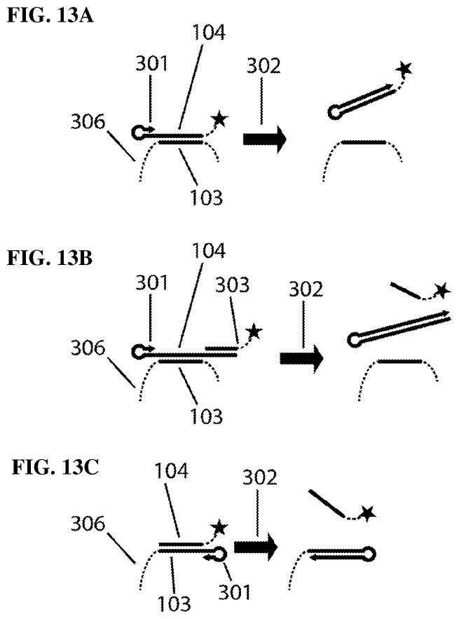

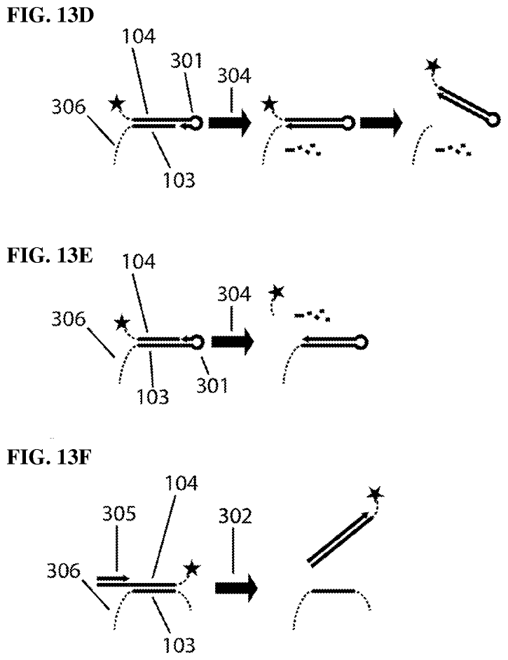

Amplification processes can make it easier and improve the results from multicolor labeling of a target or targets. In some embodiments, amplification of a nucleic acid strand (a docking strand or a primer strand) associated with a target-specific binding partner is employed to create multiple binding sites for labeled imager strands. These multiple binding sites allow for binding of multiple imager strands, which may have different labels. In one instance, the amplification is accomplished by use of a rolling circle that replicates multiple copies of a nucleic acid strand associated with the target-specific binding partner (such as an antibody) (FIG. 13D). In some embodiments, the nucleic acid strand to be replicated may have only one domain for binding a complementary imager strand. In some embodiments, the nucleic acid strand to be replicated may have more than one domain for binding respective complementary imager strands. When a nucleic acid strand has more than one domain for binding imager strands, the binding of imager strands is noncompetitive. When a nucleic acid strand has only one domain for binding imager strands and if more than one imager strand is used to label the target with more than one color, the binding of imager strands is competitive. Accordingly, nucleic acid strands, serving as a binding site for complementary imager strands labeled with different labels, may be amplified simultaneously, and the signal amplification is additive in that the signal from one type of label increases without compromising or competing with the signals from the other type of label. In such an embodiment, a first imager strand with a first label may bind to a first domain on the replicated strand and a second imager strand having a different sequence and a second label may bind to a second domain on the replicated strand. In another embodiment, the dual-label multiplexing may function more similarly to single-label multiplexing where both the first and second imager strands (with their respective different labels) have a sequence that is complementary to the same domain on the replicated strand. Thus, in one embodiment the differently-labeled imager strands do not compete for binding on the replicated strand and in another embodiment, they do compete for binding on the replicated strand.

In some embodiments, the nucleic acid strand is a docking strand, and the method further comprises increasing the number of docking strands. In some embodiments, the nucleic acid strand is a primer strand, and the method further comprises associating more than one docking strand with the primer strand.

In some instances, the target-specific binding partner is linked indirectly to a docking strand, such as through a primer strand. The primer strand also may serve as a location for amplification (such as rolling circle amplification or hybridization chain reaction amplification). In some embodiment, the nucleic acid strand is a docking strand, and the method further comprises contacting the sample with a nonlinear amplifier strand having complementarity to the docking strand, and amplifying the docking strand with rolling circle amplification and contacting the sample with labeled imager strands having complementarity to the amplified docking strands.

While the docking strand being linked to the labeled imager strand indirectly via an intermediate strand, the intermediate strand may also serve an amplification function. In some embodiments, the nucleic acid strand is a docking strand, the docking strand being linked to the labeled imager strand indirectly via an intermediate strand, and the intermediate strand comprises at least two domains for amplification to increase the number of the labeled imager strands for each type of label.

C. Target-Specific Ratios of Labels

In some embodiments, the multiple labeled imager strands are capable of binding the same docking strand, but comprise a different type of label and are provided in equal amounts. In other embodiments, the multiple labeled imager strands are capable of binding the same docking strand, but comprise a different type of label and are provided in unequal amounts.

Using a combination of equal amounts for a first target and unequal amounts for different targets can help to distinguish between them. For example, a 50:50 ratio of imager strands corresponding to target C could be distinguished from both a 25:75 ratio of imager strands corresponding to target M (25% having label 1 and 75% having label 2), as well as from a 75:25 ratio of imager strands corresponding to target N (75% having label 1 and 25% having label 2). Thus, the labeled imager strands can be provided in equal amounts, meaning that the signal provided by each labeled imager strand is the same or approximately the same (no more than 1%, 2%, 3%, 4%, or 5% difference in signal or the amount of labeled imager strand provided). The labeled imager strands can also be provided in unequal amounts, wherein the unequal amounts generate a difference in signal that can be evaluated by the user (with any one imager strand comprising at least 10%, 20%, 25%, 30%, 33%, 40%, 50%, 60%, 66%, 75%, 80%, or 90% of the population of imager strands).

A larger collection of ratios could distinguish even more targets. Likewise, using mixtures of three colors for a single target could also provide additional options for expanding the number of targets that can be labeled with a set number of fluorophore colors. In some embodiments, if a target appears throughout the sample at a high concentration or is expressed at a low level, the user can reserve a single-color channel for that target only. In other embodiments, having a number of targets assigned to a single-color channel can expand the number of targets that can be multiplexed with a given number of fluorophores.

This method may be used to qualitatively detect the presence or absence of more targets than spectral channels. It can also, however, also be used to quantitatively detect the amount of a given target by evaluating the relative amount of signal in each of the spectral channels.

For example, a 5-channel system may be used to detect 7 different targets using a dual-color labeling system with the 50:50 ratios of two labels for most of the targets (except target G) as shown in Table 4 below.

TABLE 4
7 targets imaged using 5 channels

| Targets | Label 1 | Label 2 | Label 3 | Label 4 | Label 5 |
|---|---|---|---|---|---|
| A | X | X | — | — | — |
| B | X | — | X | — | — |
| C | X | — | — | X | — |
| D | — | X | X | — | — |
| E | — | X | — | X | — |
| F | — | — | X | X | — |
| G | — | — | — | — | X |

According, when the emission is decoded in a plurality of single pixels, the level of targets is determined per each pixel, as shown in Table 5 below:

TABLE 5
Examples of the decoded levels of 7 targets
for a plurality of single pixels (each row represents
example measurements of a single pixel)

| Pixel Intensity | | | | | |
|---|---|---|---|---|---|
| By Channel | | | | | Decoded Target Level |
| 1 | 2 | 3 | 4 | 5 | (assuming 50:50 dual-color markers) |
| 4 | 1 | 1 | 1 | 10 | Target A, B, C, all present in equal level, |
| | | | | | plus Target G at level 10 |
| 7 | 4 | 1 | 1 | 3 | Target A is 4 times more than |
| | | | | | B & C which are all equal, |
| | | | | | Target G at level 3 |
| 0 | 1 | 1 | 0 | 0 | Target D detected at level 1 |
| 0 | 4 | 1 | 3 | 0 | Target D at level 1, Target E at level 3 |
| 0 | 0 | 5 | 5 | 2 | Target F at level 5, Target G at level 2 |

In another embodiment, a 5-channel dual-color labeling system is used to detect 10 different targets.

TABLE 6
10-Plex, Dual-Color Labeling, 5-Channel
System with examples of decoded level
determined for a plurality of single pixels

| | Channels | | | | |
|---|---|---|---|---|---|
| Targets | A | B | C | D | E |
| 1 | X | X | | | |
| 2 | X | | X | | |
| 3 | X | | | X | |
| 4 | X | | | | X |
| 5 | | X | X | | |
| 6 | | X | | X | |
| 7 | | X | | | X |
| 8 | | | X | X | |
| 9 | | | X | | X |
| 10 | | | | X | X |

| Pixel | | | | | Decoded Target Level |
|---|---|---|---|---|---|
| Intensity By | | | | | (assuming 50:50 dual- |
| Channel | | | | | color markers) |
| 4 | 1 | 1 | 1 | 1 | Target 1, 2, 3, 4 all |
| | | | | | present in equal level |
| 7 | 4 | 1 | 1 | 1 | Target 1 is 4 times more |
| | | | | | than the others which are |
| | | | | | all equal |
| 0 | 1 | 1 | 0 | 0 | Target 5 detected at level 1 |
| 0 | 4 | 1 | 3 | 0 | Target 5 at level 1, Target 6 |
| | | | | | at level 3 |
| 4 | 4 | 4 | 4 | 4 | All targets in equal level, |
| | | | | | OR Targets 1 at level 4, |
| | | | | | Target 8 and 9 at level 2, etc. |

In another embodiment a 4-channel system is used to detect 15 different targets using the following approach.

TABLE 7
15-Plex, Dual-Color
Labeling, 4-Channel System

| | Channels | | | |
|---|---|---|---|---|
| Targets | A | B | C | D |
| 1 | X | | | |
| 2 | X | X | | |
| 3 | X | | X | |
| 4 | X | | | X |
| 5 | X | X | X | |
| 6 | X | X | | X |
| 7 | X | | X | X |
| 8 | X | X | X | X |
| 9 | | X | | |
| 10 | | X | X | |
| 11 | | X | | X |
| 12 | | X | X | X |
| 13 | | | X | |
| 14 | | | X | X |
| 15 | | | | X |

| Pixel | | | | Decoded |
|---|---|---|---|---|
| Intensity | | | | Target Level |
| By | | | | (assuming 50:50 |
| Channel | | | | dual-color markers) |
| 3 | 0 | 0 | 0 | Target 1 at level 3 |
| 1 | 1 | 1 | 1 | Target 1 at level 1, |
| | | | | Target 9 at level 1, |
| | | | | Target 14 at level 1, |
| | | | | Target 15 at level 1 |
| | | | | OR Target 8 at level |
| | | | | 1 OR Target 2 at |
| | | | | level 1, Target 14 |
| | | | | at level 1, etc. |

D. Decoding

In some embodiments, imaging the sample to detect the labeled imager strands further comprises obtaining fluorescent spectral data from at least one image where each pixel contains the measured intensity in N_{ch} spectral channels for corresponding N_{ch} labels; and decoding the image to provide decoded images of the Nₘ targets.

In some embodiments, decoding is conducted by processing the fluorescent spectral data with N_{ch} spectral channels pixel-by-pixel,
1) determining relative intensities in each spectral channel of a label or labels for each target;
2) based on the determined relative intensities, grouping the targets into N_{ch}, mutually exclusive basis sets, the targets being the members of each basis set commonly having non-zero intensity in one of N_{ch} channels;
3) given the relative spectral intensity of the members of each basis set, and given the measured intensity of the sample in each pixel in each channel, adjusting the levels of member of each basis set to produce the least error in matching the basis set to the measured intensities; and
4) selecting the basis set with the least error and assigning each element of an output array with Nₘ values to the levels determined for the members of the selected set or to zero for members not of the selected set.

The steps (3)-(4) may be repeated for a portion or all of the pixels of the input image, and may be conducted in parallel for multiple pixels.

In some embodiments, instead of the above method based on scoring the least error in matching each basis set with the data for each pixel, other data fitting methods, such as a least-squares fit or similar parameter optimization, known in the art may be used.

In some embodiment, the relative intensities in each spectral channel for a label or labels in step (1) are determined by capturing an image of a calibration sample containing multiple labeled imager strands for a same target, and obtaining the relative intensities in spectral channels; repeating the same for the multiple labeled imager strands associated with different targets. In some embodiment, the relative intensities in each spectral channel of a label or labels for each target in step (1) are determined to meet the following conditions:
1) for each target, the sum of the relative intensities in each N_{ch} spectral channels is 1;
2) when the target is associated with only one type of label N_{ch}, the relative intensity in corresponding spectral channel N_{ch} is 1.0 and the relative intensities of the other channels are 0;
3) when the target is associated with more than one type of label, the relative intensities of each of the two spectral channels are non-zero values that sums up to 1.0, and the relative intensities of the other channels are 0; and
4) for each basis set N_{ch}, where N_{ch} is the channel number, all of the labeled imager strands include a common type of label having emission in channel N_{ch}.

To decode the images obtained with respect to spectral channel (corresponding to each label).
1) the captured fluorescent image where each pixel contains the measured fluorescent light intensity in N spectral channels and
2) a table, such as the following Table 8, which defines the encoding scheme and the relative intensities, R, from the multicolor labeled targets in each channel.

TABLE 8
Encoding Scheme and Relative Intensities of Multicolor Probes

| | Basis | Channel | | | | | |
|---|---|---|---|---|---|---|---|
| Target | set | 1 | 2 | 3 | . . . | N_{ch}-1 | N_{ch} |
| 1 | 1 | R₁₁ | R₁₂ | — | | — | — |
| 2 | 1 | R₂₁ | — | R₂₃ | | | |
| 3 | 1 | R₃₁(=1.0) | — | — | | — | — |
| | | . . . | | | | | |
| Nₘ-2 | N_{ch}-1 | — | — | — | | R_{(Nm-2)(Nch-1)} | R_{(Nm-2)(Nch)} |
| Nₘ-1 | N_{ch}-1 | — | — | — | | R_{(Nm-1)(Nch-1)} | — |
| Nₘ | N_{ch} | — | — | — | | — | R_{(Nm)(Nch)} (=1.0) |

The values in each row of the table sums to 1.0. Single-color labeled targets are indicated by a value of 1.0 in any one channel. Multicolor labeled targets will have two or more non-zero values.

In some embodiment, the values for the relative intensities R of a two-color target can be obtained as follows: a calibration image is obtained from a sample that has been stained with just the target specific binding partner having a docking strand corresponding to the two-color (two spectral channel) imager strand alone and imaged under the same conditions as will be used for subsequent experiments. Then, the intensities from each spectral channel are measured. the image of one channel of the calibration image is divided by the other to find a ratio image. Next, a mask image is generated, which selects pixels in the calibration image wherever the intensity in the two channels is above a threshold value (e.g., 20% of the maximum brightness for each pixel). Finally, the mean (or median) value, μ, of the ratio image at most or all pixels in the mask image is calculated.

The relative intensities, R₁ and R₂, in the two channels, are calculate based on the following equations:

R₁/R₂=μ

R₁+R₂=1

Solving the first equation for R₁ we have R₁=R₂μ, and by substitution in the second

R₂μ+R₂=1

or

R₂=1/(1+μ)

And, given that R₁+R₂=1, we have for R₁

R₁=1−1/(1+μ)

This calibration image, used to measure the value of for each two-color probe, only needs to be captured once for an entire run of samples prepared under the same conditions.

At initialization, in the relative intensity table, the targets are separated into subgroups. The first group contains all reagents that have emission in Channel 1 (i.e., non-zero value in column 1). Of the remaining reagents, a second group contains those that have emission in Channel 2, and of the remaining, a third group contains that have emission in Channel 3. We call each of these groups a basis set and number the basis sets 1 through N_{ch} where N_{ch} is the channel number (as shown in Table 1).

One example of processing, the fluorescent spectral data pixel-by-pixel of the input image is as follows. For each pixel, we perform the following steps:
(1) For each basis set N_{ch}, where N_{ch} is the channel number, initialize an array of values to the measured pixel intensities in each channel and call this array the basis set error array.
(2) For each dual color-labeled target of the basis set b with emission in two channels N_{b} and Nᵢ, find the intensity of the pixel in channel Nᵢ and use the known ratios of the dual color reagent to estimate its contribution to the emission in channel N_{b} (the basis channel).

TABLE 9
Exemplar relative intensity
ratios in spectral channels

| Target | Channel 1 | Channel 2 | Channel 3 |
|---|---|---|---|
| A | 0.6 | 0.4 | 0.0 |
| B | 0.25 | 0.0 | 0.75 |
| C | 1.0 | 0.0 | 0.0 |
| D | 0.0 | 0.5 | 0.5 |
| E | 0.0 | 1.0 | 0.0 |
| F | 0.0 | 0.0 | 1.0 |

For example, in the table above, Target B is 3 times brighter in Channel 3 than in Channel 1 (3.0=7.5/0.25), so if the Channel 3 intensity is 1200, the channel 1 intensity of the target is estimated to be 400 (=1200/3.0).

The method further comprises: (3) Subtract the expected contribution of this dual color-labeled target from the basis set error array for the basis channel N_{b} and set the error array element for channel Nᵢ to zero; (4) Repeat, starting at step (2), for each of the dual color marker in the basis set; (5) If after all of the dual color reagents have been processed (i.e., there is remaining signal), and the basis set error array for channel N_{b} is >0, and the basis set contains a single color reagent, assign that intensity to the single-color reagent in the basis set and set the error array element for channel N_{b} to zero. If there is no single color reagent in the basis set, the basis set error is not adjusted; (6) calculate the score for this basis set by summing the absolute values of each element of the basis set error array (i.e. the score across all channels); (7) Repeat steps (1)-(6) above for each of the basis sets to calculate the score for each of the basis sets.

(8) After all the basis sets have been processed, select the basis set with the lowest absolute error for this pixel location and set the output images for all the targets in that basis set to the intensities found in steps (2) to (6) for the selected basis set; (9) Set the output images for the targets not in the selected basis set to zero.

The above steps (1)-(9) are repeated for a portion or all of the pixels in the input image sequentially or in parallel.

In some embodiments, instead of the simple sum of the absolute error of each basis set in each channel used here, one could use an alternative scoring metric such as mean square error. Further, instead of the above method based on scoring the least error of each basis set, other data fitting methods, such as a least-squares fit or similar parameter optimization, known in the art may be used.

In some embodiment, a system for detecting a plurality of target molecules from spectral fluorescence data is provided and is capable for decoding the fluorescence data in N_{ch} spectral channels (obtained with N_{ch} labels) to detect the location and quantify Nₘ targets wherein Nₘ is larger than N_{ch}. The system may comprise a fluorescent microscope, a light source, a detection stage, one or more processors, a memory, and one or more programs stored in the memory, wherein the one or more programs are configured to be executed by the one or more processors, and wherein the one or more programs include instructions for the above-described image acquisition, encoding, and decoding steps. In some embodiments, the one or more programs include instructions for: (1) determining relative intensities in each spectral channel of a label or labels for each target; (2) based on the determined relative intensities, grouping the targets into N_{ch}, mutually exclusive basis sets, the targets being the members of each basis set commonly having non-zero intensity in one of N_{ch} channels; (3) given the relative spectral intensity of the members of each basis set, and given the measured intensity of the sample in each pixel in each channel, adjusting the levels of member of each basis set to produce the least error in matching the basis set to the measured intensities; and (4) selecting the basis set with the least error and assigning each element of an output array with Nₘ values to the levels determined for the members of the selected set or to zero for members not of the selected set.

In some embodiments, steps (3)-(4) are repeated for a portion of pixels of the input image. In some embodiments, steps (3)-(4) are repeated for all of pixels in the input image. In some embodiments, steps (3)-(4) are conducted in parallel for multiple pixels of the input image.

In some embodiments, the relative intensities in each spectral channel for a label or labels in step (1) are determined by capturing an image of a calibration sample containing multiple labeled imager strands for a same target, and obtaining the relative intensities in spectral channels; repeating the same for the multiple labeled imager strands associated with different targets.

In some embodiments, the relative intensities in each spectral channel of a label or labels for each target in step (1) are determined to meet the following: 1) for each target, the sum of the relative intensities in each N_{ch} spectral channels is 1; 2) when the target is associated with only one type of label N_{ch}, the relative intensity in corresponding spectral channel N_{ch} is 1.0 and the relative intensities of the other channels are 0; 3) when the target is associated with more than one type of label, the relative intensities of each of the two spectral channels are non-zero values that sums up to 1.0, and the relative intensities of the other channels are 0; and 4) for each basis set N_{ch}, where N_{ch} is the channel number, all of the labeled imager strands include a common type of label having emission in channel N_{ch}.

In some embodiments, the R values that represent the relative intensities of the more than one label is determined by: 1) obtaining a calibration image of a sample which has been stained with just the multiple imager strands with two different types of labels alone and imaged under the same conditions as will be used for subsequent experiments; 2) measuring the intensities from each spectral channel; 3) dividing the image of one channel of the calibration image by the other to find a ratio image; 4) creating a mask image that selects pixels in the calibration image wherever the intensity in the two channels is above a simple threshold (e.g. the threshold might be 20% of the maximum brightness for each); 5) calculating the mean or median value, μ, of the ratio image at most or all pixels in the mask image; and 6) calculating the relative intensities, R₁ and R₂, in the two channels having the following relationships:

R₁/R₂=μ

R₁+R₂=1

Background signals In fluorescence imaging, the background signals may be produced by a number of possible sources including: 1) excitation/emission filter out-of-band transmission, 2) autofluorescence in the sample, 3) non-specific binding of the reagents, 4) image artifacts (dust, air bubbles, etc.), and/or 5) detection system background noise, and other sources. These background signals violate the encoding scheme, are often present across all channels, and have nothing to do with the multicolor encoding used in the staining. To mitigate their effect, the method may further comprise preprocessing the input images by subtracting a best estimate of the background intensity at each pixel. The background intensity may be estimated from an image of a similar sample scanning without any staining or label (to measure autofluorescence and detection system background) and/or by analyzing the histogram of the input marker images masked for regions with DAPI emission (to distinguish pixels associated with the sample rather than the background) for large populations of the lowest intensity values (assuming the signal pixels are fewer in number than the background pixels).

The image of the basis set error in each pixel can be used as a confidence map to define areas of the image with good decoding from areas where the decoding was not as successful. The image of the basis set index at each pixel can also serve as a map of regions with different biological function since the basis sets are often grouped this way.

### II. Components of the Exchange Imaging Method

A. Target-Specific Binding Partners

The target recognition moiety refers to antibodies and antibody-like molecules that can be used to detect the target molecule. Antibody refers to any immunoglobulin from any species that can specifically recognize a target molecule. Antibody-like molecule refers to (Class A) any engineered variation or fragment of an antibody such as Fab, Fab′, F(ab′₂, single heavy chain, diabody, and the like (antigen binding fragments of antibodies) (Class B) any known binding partner of a target molecule and engineered variants of such binding partner, (Class C) any binding partner of the target molecule engineered via directed evolution (e.g., peptides and aptamers), and (Class D) any molecule that selectively forms covalent bond(s) with a target (e.g., a suicide substrate of an enzyme of interest).

The target-specific binding partner may be provided in a liquid medium or buffer solution. Table 10 provides a representative listing of targets and corresponding target recognition moieties.

TABLE 10
Representative Targets and Target Recognition Moieties

| | Target Recognition | |
|---|---|---|
| Target | Moiety | Source or Sequence |
| Any protein | Antibody (Class A) | Variable |
| Fluorescein | Antibody (Class A) | Abcam, product # |
| (chemical compound) | | ab7253 |
| Digoxigenin | Antibody (Class A) | Abcam, product # |
| (chemical compound) | | ab76907 |
| Biotin | Avidin/Streptavidin | |
| | (Class B) | |
| Epidermal growth | Epidermal | |
| factor receptor | growth factor | |
| (EGFR, protein) | (EGF, Class B) | |
| Platelet-derived | Platelet-derived | |
| growth factor | growth factor | |
| receptor | (PDGF, Class B) | |
| (PDGFR, protein) | | |
| Epidermal growth | E07 aptamer | Li et al., PloS ONE, |
| factor receptor | (Class C) | 2011;6(6):e20299 |
| (EGFR, protein) | | |
| Integrins (protein) | RGD-containing | |
| | peptides (Class B) | |
| TNF-α (protein) | T09.12 peptide | Xu et al., Chem Biol. |
| | (Class C) | 2002 August; 9(8):933-42. |
| HaloTag (enzyme) | Halogenated | Bioconjug Chem. 2015 |
| | compounds | June 17; 26(6):975-86. |
| | (Class D) | |
| Oxidosqualene | 3[H]29-methylidene- | Biochem Biophys Res |
| cyclase | 2,3-oxidosqualene | Commun. 1992 August |
| (OSC, enzyme) | (3[H]29-MOS, Class D) | 31;187(1):32-8. |

Table 11 provides alisting of additional targets. Antibodies and other known binding partners of these targets may be used astarget recognizing moieties.

TABLE 11
Additional Representative Targets
Actin
AIF
AKT
alpha-synuclein
amyloid precursor
protein
annexin
arrestin
BAD
BAX
Bcl-2
Bcl-2
beta-catenin
BRCA1
cAMP
caveolin
CD20
CD3
CD4
CD45
CD68
CD8
collagen
CREB
DNA
E-Cadherin
EGFR
EpCAM
ER
ERK
ERK
FOXA
FOXP3
GABA
GAPDH
GFP
granzymeB
GRB2
HER2
HER3
HIF-1
histoneH3
H5P27
HSP70
HSP90
keratin
Ki67
lamin
MAPK
MEK
MET
MMP
mTOR
MYC
NeuN
p21
p53
PAX
PD-1
PD-L1
PI3K
PR
PSD95
RAS
SOX
STAT
synapsin
Tau
TOM20
tubulin
ubiquitin
VEGF
vimentin
WNT

B. Docking Strands

In some embodiments, the docking moiety or docking strand is a nucleic acid, a protein, a peptide, or a chemical compound. Many proteins and domains of proteins are known to interact with other proteins, domains or peptides. Some of the best-known domains include SH2, SH3, and WD40 domains. In many cases the binding partner of these proteins and domains are known and can be engineered to have the desired affinity. For example, biotin and avidin/streptavidin interact with sufficient specificity. Many other chemical compounds, such as digoxigenin, fluorescein, tacrolimus and rapamycin also have well known binding partners.

In some embodiments, the docking strand comprises nucleic acids.

In some embodiments, the nucleic acids are single stranded nucleic acids such as single stranded DNA, RNA, or a nucleic acid analog. A nucleic acid analog (also known as non-natural nucleic acid) may include an altered phosphate backbone, an altered pentose sugar, and/or altered nucleobases. Nucleic acid analogs may include, but are not limited to, 2′-O-Methyl ribonucleic acid, 2′-fluoro ribonucleic acid, peptide nucleic acid, morpholino and locked nucleic acid, glycol nucleic acid, and threose nucleic acid.

In some embodiments, the docking strand is attached to the imager strand covalently and in other embodiments noncovalently.

In some embodiments, the docking strand comprises single-stranded nucleic acids and may be from about 5 to 20 nucleotides long, from about 8 to 15, or from about 10 to 12 nucleotides long. In some embodiments, the docking strand is about 5, 8, 9, 10, 11, 12, 13, 14, 15, 18, or 20 nucleotides long.

The docking strand may be an independent element or it may be part of the target recognizing moiety. For example, if the target recognizing moiety is an antibody, part of the Fc domain of the antibody may be the docking strand and a peptide or protein that binds the Fc domain may be used, such as protein A or protein G.

The docking strand may be provided in a liquid medium or buffer solution.

C. Imager Strands

In some embodiments, the docking strand may be a nucleic acid strand. In such cases, the observable moiety or label may be conjugated to an imager moiety, which may be a nucleic acid strand that is complementary to the docking strand. In other words, the imager strand specifically binds the docking strand. In such a case, the label may be conjugated to an imager moiety that may be from about 5 to 20 nucleotides long, from about 8 to 15, or from about 10 to 12 nucleotides long. In some embodiments, the imager moiety is about 5, 8, 9, 10, 11, 12, 13, 14, 15, 18, or 20 nucleotides long.

In some embodiments, the imager strand is even longer, such as from 20 to 80 nucleotides long, for example less than or equal to 80, 75, 70, 65, 60, 55, 50, 45, 40, 35, or 30 nucleotides long. In embodiments employing a hairpin structure for the imager strand, the length of the imager strand may be longer than if no hairpin structure is used.

In some embodiments, the complementary portions between the imager moiety and the docking strand may be from about 5 to 20 nucleotides long, from about 8 to 15, or from about 10 to 12 nucleotides long. In some embodiments, the complementary portions between the imager moiety and the docking strand may be about 5, 8, 9, 10, 11, 12, 13, 14, 15, 18, or 20 nucleotides long.

In some embodiments, the nucleic acid imager strand comprises single stranded nucleic acids such as single stranded DNA, RNA, or a nucleic acid analog. A nucleic acid analog (also known as non-natural nucleic acid) may include an altered phosphate backbone, an altered pentose sugar, and/or altered nucleobases. Nucleic acid analogs may include, but are not limited to, 2′-O-Methyl ribonucleic acid, 2′-fluoro ribonucleic acid, peptide nucleic acid, morpholino and locked nucleic acid, glycol nucleic acid, and threose nucleic acid.

In some embodiments, the imager moiety is a protein, peptide, or a chemical compound, as a partner to the docking strand options discussed above in Section II.B above.

In some embodiments, the docking strand may bind to the imager moiety indirectly, such as through an intermediate moiety. For instance, when the docking strand and the imager moiety are nucleic acids, an intermediate moiety comprising nucleic acids may be used as long as the intermediate moiety has a first region complementary to the docking strand and a second region complementary to the imager moiety. In this embodiment, it is not necessary for the docking strand to be complementary to the imager moiety. The intermediate moiety may serve only a bridging function or it may also serve an amplification function.

The imager strand may be provided in a liquid medium or buffer solution.

D. Primer Strands

In some instances, the target-specific binding partner is linked indirectly to a docking strand, such as through a primer. For instance, when the docking moiety and the imager moiety comprise nucleic acids, the primer strand comprising nucleic acids may be used as a binding location for the docking strand (if the docking strand has a region complementary to the primer strand) or it may be used as a primer for nucleic acid synthesis through, for example, rolling circle amplification. The primer strand may also be used to initiate the cascade of binding events in hybridization chain reaction amplification. In instances where the primer serves as a location for amplification (such as rolling circle amplification, hybridization chain reaction amplification), the primer is not necessarily complementary to the docking strand. Instead, it serves as a template for amplification and the docking strands are included through the amplification process.

In some embodiments, the target-specific binding partner and linked primer are added to the sample as a first step, docking strand added as a second step, and imager strand added as a third step. In another embodiment, the components are not added in discrete steps. Washing steps may be added between the first, second, and/or third steps.

In some embodiments, the primer strand comprises nucleic acids. In some embodiments, the nucleic acids are single stranded nucleic acids such as single stranded DNA, RNA, or a nucleic acid analog. A nucleic acid analog (also known as non-natural nucleic acid) may include an altered phosphate backbone, an altered pentose sugar, and/or altered nucleobases. Nucleic acid analogs may include, but are not limited to, 2′-O-Methyl ribonucleic acid, 2′-fluoro ribonucleic acid, peptide nucleic acid, morpholino and locked nucleic acid, glycol nucleic acid, and threose nucleic acid.

In some embodiments, the primer strand comprises single-stranded nucleic acids and may be from about 5 to 20 nucleotides long, from about 8 to 15, or from about 10 to 12 nucleotides long. In some embodiments, the primer strand is about 5, 8, 9, 10, 11, 12, 13, 14, 15, 18, 20, 25, 30, 35, 40, 45, 50, 55, 60, 65, or 70 nucleotides long.

The primer strand may be provided in a liquid medium or buffer solution.

E. Intermediate Strands

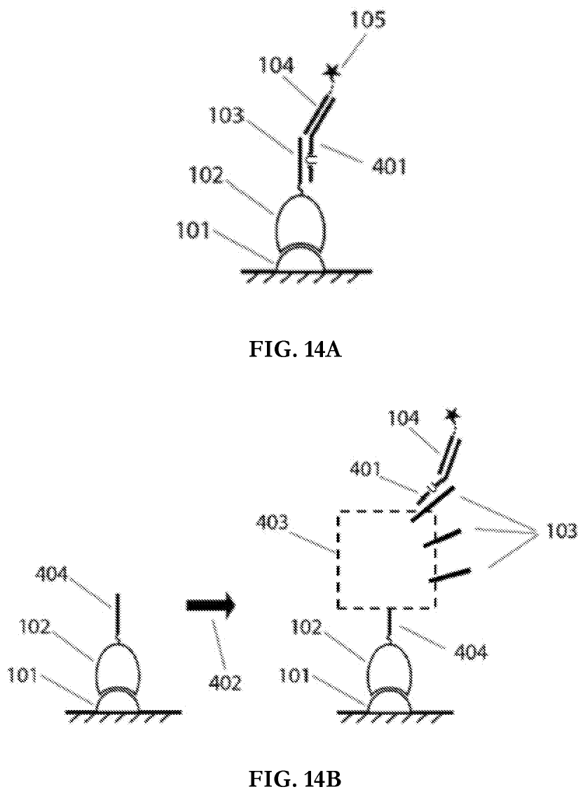

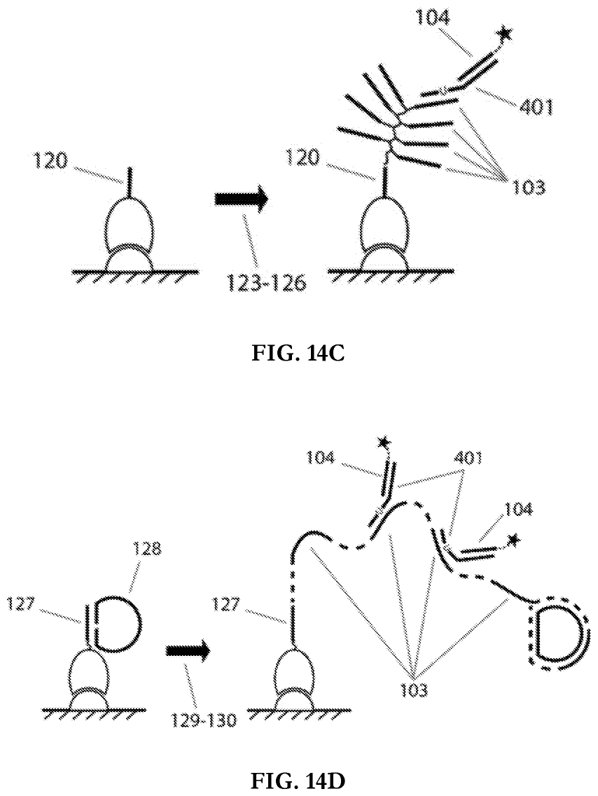

In some instances, the docking strand binds to the imager strand through an intermediate moiety (or intermediate strand). For instance, when the docking moiety and the imager moiety comprise nucleic acids, the intermediate strand comprising nucleic acids may be used as long as the intermediate strand has a first region complementary to the docking strand and a second region complementary to the imager strand. In such embodiments, it is not necessary for the docking strand to be complementary to the imager moiety. FIG. 14A shows DNA-Exchange imaging with the use of an intermediate strand (401) to link an imager strand and a docking strand bound to a target through a target-recognition moiety.

In some embodiments, the intermediate strand is added as a first step to a sample comprising the target-specific binding partner linked to a docking strand, either directly or indirectly, and the imager strands added as a second step. In another embodiment, the intermediate strand and imager strand are not added in discrete steps.

In some instances, the intermediate strand and imager strand are hybridized together before being added in a single step.

In some embodiments, the intermediate strand comprises nucleic acids. In some embodiments, the nucleic acids are single stranded nucleic acids such as single stranded DNA, RNA, or a nucleic acid analog. A nucleic acid analog (also known as non-natural nucleic acid) may include an altered phosphate backbone, an altered pentose sugar, and/or altered nucleobases. Nucleic acid analogs may include, but are not limited to, 2′-O-Methyl ribonucleic acid, 2′-fluoro ribonucleic acid, peptide nucleic acid, morpholino and locked nucleic acid, glycol nucleic acid, and threose nucleic acid.

In some embodiments, the intermediate strand comprises single-stranded nucleic acids and may be from about 5 to 30 nucleotides long, from about 8 to 15, or from about 10 to 12 nucleotides long. In some embodiments, the intermediate strand is about 5, 8, 9, 10, 11, 12, 13, 14, 15, 18, 20, 25, or 30 nucleotides long.

The intermediate strand may be provided in a liquid medium or buffer solution.

F. Nucleic Acids in Hairpin Format

Any of the linear nucleic acids described herein may optionally be provided in a hairpin format. This includes the imager strand, docking strand, primer strand, and intermediate strand. In the hairpin format, a region of from at least 1-5 nucleotides at the end of the hairpin stem region may optionally comprise only G's and C's. This G/C region is known as a clamp. The G/C region prevents or reduces fraying at the end of the hairpin to prevent opening up into linear DNA.

A hairpin may be used in contexts when a user desires to break the interaction (direct or indirect) between the imager strand and the docking strand using a polymerase with a strand-displacement activity (e.g., phi29) or a polymerase with a 5′-to-3′ exonuclease activity (e.g., DNA Polymerase I). A hairpin may also be used to limit unwanted binding of single-stranded nucleic acids.

### III. Variations in Method Steps

There are various ways of approaching multiplexed imaging, including options for amplifying the signal using amplification steps at different time points and repeated steps to allow for imaging of multiple targets or reinterrogation of a single target. Methods may also optionally include extinguishing the signaling image at various points in time.

1. Labels

Various labels, also known as observable moieties, may be bound to the imager strand. These labels or observable moieties assist the user by enabling detection of the bound imager strand. When the application refers to detecting bound labeled imager strands, the application references detecting the signal produced by the label or observable moiety bound to the imager strand.

In some embodiments, any label may be employed and, in some embodiments, the label is optically observable. The moiety may be signal absorbing or signal emitting. Of signal emitting molecules, molecules that fluoresce may be used, such as organic small molecules, including, but not limited to fluorophores, such as, but not limited to, fluorescein, Rhodamine, cyanine dyes, Alexa dyes, DyLight dyes, Atto dyes, etc.

In some embodiments, organic polymers, such as p-dots may be employed. In some embodiments, the label may be a biological molecule, including but not limited to a fluorescent protein or fluorescent nucleic acid (including fluorescent RNAs including Spinach and its derivatives). In some embodiments, the label may be an inorganic moiety including Q-dots. In some embodiments, the observable moiety may be a moiety that operates through scattering, either elastic or inelastic scattering, such as nanoparticles and Surface Enhanced Raman Spectroscopy (SERS) reporters (e.g., 4-Mercaptobenzoic acid, 2,7-mercapto-4-methylcoumarin). In some embodiments, the label may be chemiluminescence/electrochemiluminescence emitters such as ruthenium complexes and luciferases. The observable moiety may generate an optical signal, an electromagnetic signal (across the entire electromagnetic spectrum), atomic/molecular mass (e.g. detectable by mass spectrometry), tangible mass (e.g., detectable by atomic force microscope), current or voltage.

2. Fluorescent Imaging

Various types of fluorescent imaging may be used in conjunction with the methods. In some embodiments, imaging is performed using fluorescence microscopy including widefield, confocal (line and point scanning, spinning disk), total internal reflection (TIR), stimulated emission depletion (STED), light-sheet illumination (including lattice light-sheet illumination), structured illumination (SIM), and expansion microscopy.

3. Multiplexing

In exchange imaging, spectral multiplexing and sequential multiplexing can either be used alone or in conjugation with each other. Using more than one technique of multiplexing, however, can significantly increase the number of targets that a user can visualize during a particular experiment. Combining both spectral multiplexing and sequential multiplexing can increase the overall convenience of performing the imaging for the user and reduce disruption to the sample being imaged.

Spectral multiplexing does not necessitate extinguishing the signal from the first label before viewing the second label. For example, in the case of fluorophores, different excitation wavelengths of light can be used to individually excite different fluorophores. This does not require separate rounds of imaging. On the other hand, for sequential multiplexing requires extinguishing the signal from the first round of imaging before the second round of imaging

In some embodiments, multiple rounds of imaging are performed with at least some of the same fluorophores. For example, in a first round of imaging, target A can be imaged with label X, target B can be imaged with label Y, and target C can be imaged with label Z. As a next step, the signals from these labels can be extinguished. Then, in a second round of imaging, target D can be imaged with label X, target E can be imaged with label Y, and target F can be imaged with label Z. In some embodiments, at least two targets are imaged using at least two labels, the signal extinguished, and then at least one more target is imaged using at least one of the same labels, wherein the imaging steps may be performed in either order. This means that the order of steps could be reversed so the first imaging step comprises imaging at least one target, the signal extinguished, and the second imaging step comprises imaging at least two targets.

4. Amplification Methods

In microscopy, signal amplification is desired in many situations such as when the level of target is low, when the allowable exposure time is short, and/or when the sensitivity of the imaging equipment is low. Signal amplification offers advantages in DNA exchange immunofluorescence. In traditional, single-plex immunofluorescence (where only one target is analyzed), one often uses unconjugated primary antibody and fluorescent-labeled secondary antibody. Because the secondary antibodies are often polyclonal, multiple molecules of secondary antibody can bind to one molecule of primary antibody, resulting in amplification of signal. In DNA exchange immunofluorescence, however, in some embodiments, users directly label the DNA docking strand to the primary antibody, thus eliminating such signal-amplification step obtained by using a polyclonal secondary antibody. As a result, in some cases, DNA exchange immunofluorescence may have lower signal intensity relative to traditional immunofluorescence.

Thus, in one embodiment, amplification is used to improve the signal intensity in multiplexed DNA exchange immunofluorescence. Many methods for signal amplification in microscopy exist, but not all can be applied to DNA exchange immunofluorescence. One of the most well-known methods involves linking (covalently or non-covalently) the target-recognizing molecule (e.g., antibody) to an enzyme that can convert a non-observable substrate into an observable product. Many enzymes such as horseradish peroxidase (HRP), alkaline phosphatase (AP), glucose oxidase (GO), β-galactosidase (β-gal) have been used for these purposes. And an array of chromogenic, fluorogenic and chemiluminescent substrates for these enzymes have been developed, such as 3,3′-diaminobenzidine (DAB), nitro blue tetrazolium chloride (NBT), 5-bromo-4-chloro-3-indolyl phosphate (BCIP), and 5-bromo-4-chloro-3-indoyl-β-D-galactopyranoside (X-Gal). Another strategy to exploit such enzymes in signal amplification is to create covalent bonds between the target (or other molecules in vicinity to the target) and an observable reporter molecule. This strategy is exemplified by tyramide signal amplification (TSA) technology that is commercialized by Thermo Fisher and Perkin Elmer, among others. However, none of these signal-amplification methods is compatible with exchange imaging, as the observable reporter, enzyme/primer or the substrate is brought to the vicinity of the target permanently or without a decodable docking strand. DNA-based (i.e., decodable) signal amplification methods where the observable signal can, at least in principle, be removed have been reported (e.g., Zimak, et al., Chembiochem 13(18):2722-8 (2012) (PMID: 23165916)). However, such methods involve multiple rounds of manipulation and the signal gain is modest.

Another type of signal amplification involves linking (covalently or non-covalently) the target-recognizing molecule to a primer molecule of a polymerization or dendrimerization reaction. On example of such polymerization reactions is rolling circle amplification (RCA) where the primer of the RCA is linked to the target-recognizing molecule and is converted to a long repetitive single-stranded DNA. Fluorescent molecules can be either directly incorporated into the RCA product via fluorescent-labeled nucleotides, or be bound to the RCA product as a part of a fluorescent-labeled oligonucleotide that is designed to hybridize to the RCA product. Other examples of such polymerization or dendrimerization reactions include branched DNA toehold-based strand displacement (Schweller et al. PMCID: PMC3517005), hybridization chain reaction (HCR) (Dirks et al., 2014, PMID: 15492210, 24712299) and a similar DNA hairpin-based dendrimerization reaction (Yin et al., 2008, PMID 18202654), which here we call HDR. Common applications of amplification methods such as RCA, HCR, and HDR do not include the option for Exchange Imaging, but could be compatible as demonstrated by the improvements described herein.

Herein we discuss embodiments covering signal amplification that is compatible with exchange imaging. We describe a series of embodiments that make signal amplification compatible with exchange imaging. These embodiments can be divided into two classes based on whether the amplification product is decodable. For example, if the amplification product contains a docking strand component (e.g. single-stranded DNA), many (e.g., >5) antibodies against different targets can be programmed to generate such product of distinct docking strand sequences that can later be decoded by the ssDNA molecules of complementary sequence. Thus, such amplification product is considered decodable. In such cases, signal amplification for different targets can be carried out simultaneously, followed by simultaneous and/or sequential imaging of different amplification products. Simultaneous amplification carried out for different targets can be considered multiplexed amplification.

In contrast, when the amplification product is a fluorophore or label that is covalently attached or noncovalently deposited near the target but does not contain a docking strand that could interact with an imager strand, these amplification products are considered undecodable. For example, when the enzyme responsible for signal amplification is HRP, the product is a chemical chromophore that does not allow many variations that can be specifically bound by many molecules serving as imager strands. In such cases, signal amplification for different targets may be carried out sequentially, and the enzyme linked to a target that has already been amplified may be removed from the sample. Simultaneous signal amplification of undecodable amplification products is possible if orthogonal enzyme-substrate pairs can be used.

Therefore, in some embodiments a method comprises (1) contacting a sample being tested for the presence of one or more targets with one or more target-specific binding partners, wherein each target-specific binding partner is linked to a nucleic acid strand, and wherein target-specific binding partners of different specificity are linked to different nucleic acid strands, (2) optionally removing unbound target-specific binding partners, (3) wherein the nucleic acid strand is a docking strand or a primer strand and if the nucleic acid is (a) a docking strand, increasing the number of docking strands associated with each target-specific binding partner or (b) a primer strand, associating more than one docking strand with the primer strand (in either (a) or (b), such as, for example, amplifying the number of docking strands available), (4) contacting the sample with labeled imager strands capable of binding a docking strand, directly or indirectly, (5) optionally removing unbound labeled imager strands, (6) imaging the sample to detect bound labeled imager strands, (7) optionally removing the bound labeled imager strands from the docking strands, and (8) optionally repeating steps (1)-(6), or any subset thereof.

By docking strands associated each target specific binding partner, here and throughout the application, Applicant does not intend to require amplification to occur on every single docking strand, but that amplification generally occurs on the docking strands associated with the various target-specific binding partners, as desired by the user (including amplifying only some docking strands participating in detecting targets A and B, while not amplifying docking strands participating in detecting target B.) Amplification may also be incomplete, such as amplification occurring on only some but not all of the copies of the docking strand participating in detecting a given target. Additionally, amplification may replicate the entire docking strand or it may replicate only a portion of the docking strand sufficient for binding an imager strand.

Additionally, in some embodiments, a method comprises (1) contacting a sample being tested for the presence of one or more targets with one or more target-specific binding partners, wherein each target-specific binding partner is linked to a nucleic acid strand and wherein target-specific binding partners of different specificity are linked to different nucleic acid strands, (2) optionally removing unbound target-specific binding partners, (3) contacting the sample with labeled imager strands capable of binding a docking strand, directly or indirectly, wherein the nucleic acid strand in (1) is either a primer strand or a docking strand and if the nucleic acid strand is a primer strand, it is linked to a docking strand (4) optionally removing unbound labeled imager strands, (5) imaging the sample to detect bound labeled imager strands and determine if amplification (step (7)) is required, (6) optionally removing the bound labeled imager strands from the docking strands, (7) optionally increasing the number of docking strands associated with each target-specific binding partner (such as, for example, amplifying the number of docking strands available by multiple means, including, but not limited to self-assembly of docking strand complexes, other assembly methods, branched and circular docking strands, etc.), and (8) optionally repeating steps (1)-(7), or any subset thereof.

a) Amplification

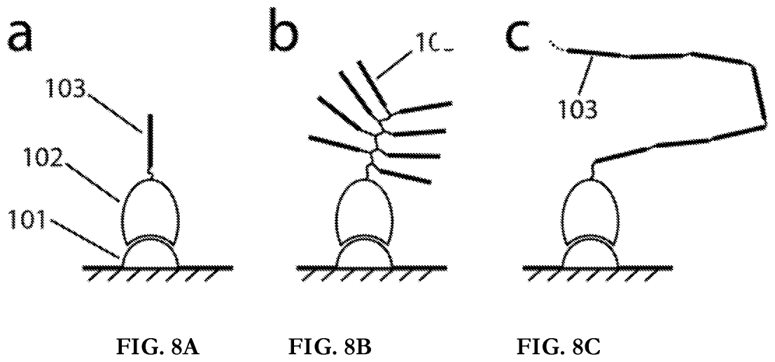

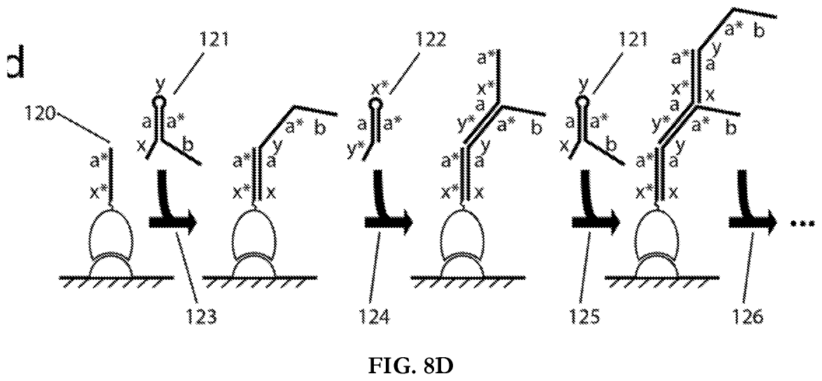

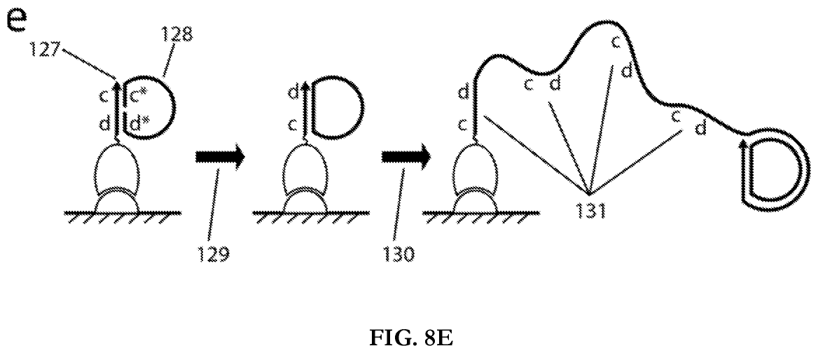

Decodable amplification products. Decodable amplification products include those cases in which the amplified product is a docking strand. In one embodiment, the docking strand does not contain an observable label. In one embodiment, the docking strand serves as a barcode for an observable label (or imager strand). It should be noted at the outset that the docking strands, or docking sites, may be introduced to the target during a signal-amplification reaction (FIGS. 8B-C), so that multiple docking strands are attached to one target molecule. Without being limiting, there are at least two strategies of achieving this: (Strategy 1) creating multiple docking sites that are attached to a scaffold, which is in turn attached to the target-recognizing moiety (FIG. 8B), and (Strategy 2) creating multiple binding sites on one piece of long single-stranded DNA that is attached to the target-recognizing moiety (FIG. 8C).

One may use RCA, HCR or HDR to generate a polymeric or dendrimeric product from the primer molecule linked to the antibody. In some embodiments, the product may contain many (e.g., greater than 2, 5, 10, 15, 20, 25, 50, 100, etc.) copies of single-stranded DNA domains that can serve as the docking strand and thus be recognized by oligonucleotides serving as the imager strand. Such DNA domains may be long enough (e.g. 7, 8, 9, 10, 11, 12, 13, 14, 15, 20, or more nucleotides long, or be able to bind its complementary strand with Kd<1 nM at imaging condition). For RCA, this is achieved regularly. For HCR and HDR, if necessary one can include, at the loop or tail of the substrate hairpin, DNA domains that do not participate in the strand-displacement cascades but constitute part or the entirety of the imager strand-binding site.

In one embodiment, a modified version of hybridization chain reaction (HCR) is employed for signal amplification, in which two hairpins (105 and 106 of FIG. 8D) are assembled onto the primer strand (104 of FIG. 8D) in an alternating fashion. Either or both of the two hairpins can carry a docking site (domain b on hairpin 105 of FIG. 8D). Tens to hundreds of hairpin units can be assembled onto one primer strand, brining tens to hundreds of docking sites to the target-recognizing moiety. Several pairs of hairpin sequences (without the docking site) have been demonstrated by the Pierce group to enable successful HCR reactions. Hairpin sequences with the docking sites can be designed with the same principle, although care may be taken to ensure that the docking site does not form unwanted secondary structure with the rest of the hairpin.

In another embodiment, signal amplification involves linking (covalently or non-covalently) the target-recognizing molecule to a primer molecule of a polymerization or dendrimerization reaction. On example of such polymerization reactions is rolling circle amplification (RCA, FIG. 8E) where the primer of the RCA is linked to the target-recognizing molecule and is converted to a long repetitive single-stranded DNA. Fluorescent molecules can be either directly incorporated into the RCA product via fluorescent-labeled nucleotides, or be bound to the RCA product as a part of a fluorescent-labeled oligonucleotide that is designed to hybridize to the RCA product.

There are many ways to carry out RCA, one of which is to first ensure that the oligonucleotide conjugated to the target-recognizing moiety (here we call ‘primer’, 127 of FIG. 8E) has an extendable 3′ end. Then one can introduce a linear template strand (128 of FIG. 8E) that can hybridize to the primer in the circular fashion, in which the primer brings the two ends of the template together so that the two ends can be ligated. Next, a ligase (such as T4 DNA ligase or CircLigase™ ssLigase, for example) is used to ligate the two ends to form a circle. After the ligation the primer is hybridized to the circular template. Next, a DNA polymerase with strand-displacement activity (e.g., phi29, Bst, Vent(exo)) can extend the primer along the circular template multiple rounds to create a concatemeric repeat. Part of the entirety of the repeat unit (domains c-d, or 131 of FIG. 8E) can serve as the docking sites (or docking strands) for imager strands.

An alternative method of RCA involves the use of a nonlinear amplifier or template strand, wherein an oligonucleotide (such as a docking strand) conjugated to the target-recognizing moiety is hybridized to a circular DNA template (amplifier strand), followed by extension of the docking strand by a DNA polymerase to create a concatemeric repeat of the reverse complement of the amplifier strand (i.e. an amplified strand or RCA product). The hybridization of the amplifier strand to the oligonucleotide conjugated target-recognizing moiety may occur before (preassembly or prehybridization) or after the oligonucleotide conjugated target-recognizing moiety contacts the sample.

FIG. 14B illustrates a primer strand (404) used to amplify the number of docking strands associated with a target-binding complex, where the resulting amplified product (403) is attached to multiple docking sites (103) and can be imaged with an imager strand, directly or indirectly through an intermediate strand as shown. FIG. 14C shows amplification of the number of docking strands associated with a target using a primer strand to initiate a hybridization chain reaction and imaging with the addition of an imager strand, bound to docking strand through an intermediate strand. FIG. 14D shows amplification of the number of docking strands associated with a target using a primer strand as a template for ligation and rolling circle amplification, followed by the addition of an imager strand, bound to docking strands through intermediate strands for imaging.

Thus, in some embodiments, at least one oligonucleotide-conjugated target-recognizing moiety is hybridized to a nonlinear amplifier strand before being introduced to the sample. When the user chooses to pre-assemble an antibody-DNA conjugate with an amplifier, a method to test a sample for the presence of one or more targets comprises (1) contacting a sample being tested for the presence of one or more targets with one or more target-specific binding partners, wherein each target-specific binding partner is linked to a nucleic acid strand and wherein target-specific binding partners of different specificity are linked to different nucleic acid strands, and wherein at least one nucleic acid strand is hybridized to a nonlinear amplifier strand (2) optionally removing unbound target-specific binding partners, (3) amplifying the docking strand with rolling circle amplification (i.e., increasing the number of docking strands or introducing a plurality of docking strands) to produce a rolling circle amplification product (4) contacting the sample with labeled imager strands having complementarity to the docking strand or the rolling circle amplification product, (5) imaging the sample to detect bound labeled imager strands, (6) optionally removing the bound labeled imager strands, and (7) optionally repeating steps (1)-(7), or any subset thereof. In this process, the rolling circle amplification product comprises a concatemeric repeat of the reverse complement of the amplifier strand.

In another embodiment, imager strands may be hybridized to the RCA product (e.g. the concatemeric repeat of the reverse complement of the amplifier strand that is linked to the target-recognizing moiety) during the RCA reaction. In some embodiments, therefore, amplification occurs using rolling circle amplification, while in the presence of labeled imager strands having complementarity to the amplified strand. For example, a sample may be contacted with an oligonucleotide conjugated to a target-recognizing moiety that is either prehybridized to an amplifier strand or the amplifier strand may be hybridized in a later step. Then, all additional components for the RCA reaction may be added in one step including proteins (e.g. DNA polymerases, optionally BSA), nucleotides, buffer solution, salts, and imager strands. In some embodiments, a user may wish to prevent the imager strand from being amplified. This can be accomplished by several means, including, but not limited to employing a 3′-modified imager strand having a modification on the 3′ end. For example, the 3′ modification on the imager strand may include a label (such as a fluorophore), a modified base, a stop code or terminator, a 3′-O-modification, a dideoxy-C, a dideoxy-G, a dideoxy-A, a dideoxy-T, an inverted nucleotide, any modification that eliminates the presence of a 3′ hydroxyl group, or a single-stranded extension of the 3′ end that is not complimentary to the amplifier strand.

In addition to HCR and RCA, other examples of such polymerization or dendrimerization reactions include DNA hairpin-based dendrimerization reaction (HDR) (Yin et al., 2008, PMID 18202654), and toe-hold mediated strand displacement.

DNA strand displacement is a method for the isothermal and dynamic exchange of DNA complexes. Strand displacement can be designed and intentionally controlled based on an understanding of DNA hybridization interactions and thermodynamics, and can be facilitated by introducing engineered handles which are known as “toehold domains.” The ability to modulate binding interactions and exchange hybridization partners gives rise to a series of potential signal amplification applications.

In another embodiment, an encodable tyramide-based signal amplification product is described. This method comprises (1) contacting a sample being tested for the presence of one or more targets with one or more target-specific binding partners, wherein each target-specific binding partner is linked to a nucleic acid strand and wherein target-specific binding partners of different specificity are linked to different nucleic acid strands, (2) optionally removing unbound target-specific binding partners, (3) contacting the sample with enzyme-labeled strands capable of binding a docking strand, wherein the nucleic acid strand in (1) is either a primer strand or a docking strand and if the nucleic acid strand is a primer strand, it is linked to a docking strand (5) optionally removing unbound enzyme-labeled strands, (4) contacting the sample with tyramide-bound docking strands, (5) enzymatically converting the tyramide moiety into an activated state, wherein the activated state results in a covalent linkage of the tyramide-bound docking strand to the enzyme-labeled target site, (6) optionally quenching the enzymatic reaction, (7) removing the enzyme-labeled strands, and (8) optionally repeating a subset of steps 3-8. In one embodiment, the enzyme-linked strand is an HRP-linked strand.

In another embodiment, a method comprises (1) contacting a sample being tested for the presence of one or more targets with one target-specific binding partner, wherein the target-specific binding partner is linked to an enzyme (2) optionally removing unbound target-specific binding partners, (3) contacting the sample with tyramide-bound docking strands, (4) enzymatically converting the tyramide moiety into an activated state, wherein the activated state results in a covalent linkage of the tyramide-bound docking strand to the enzyme-labeled target site, (5) quenching the enzymatic reaction, and (6) optionally repeating a subset of steps 1-8, wherein target-specific binding partners of different specificity are introduced. In one embodiment, the enzyme-linked target-specific binding partners contain HRP.

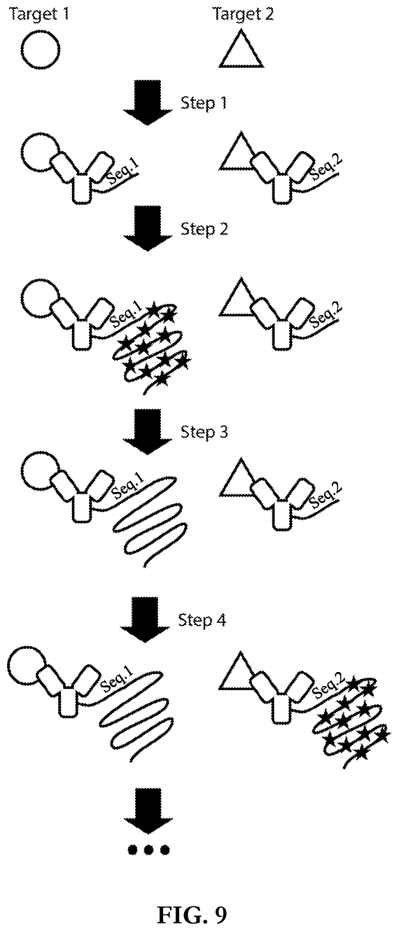

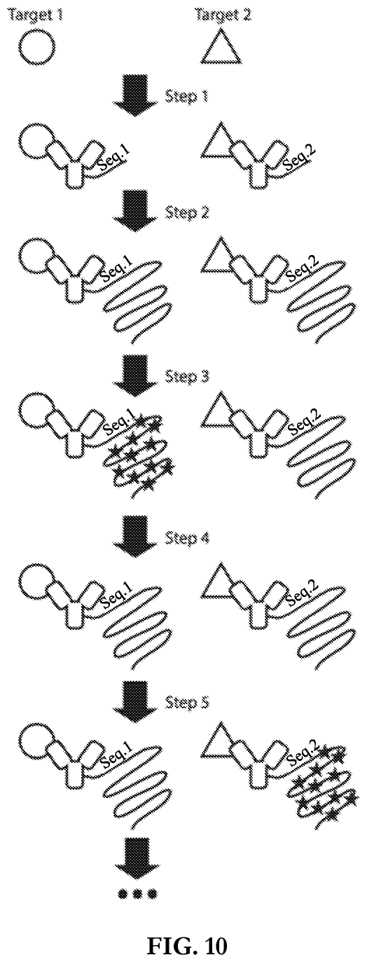

As shown in FIG. 9, the amplification of multiple targets can be carried out sequentially. Alternatively, the amplification of multiple targets can be carried out simultaneously (FIG. 10). Imaging steps can be carried out between rounds of amplification, or following all rounds of amplification.

Undecodable amplification products. Undecodable amplification products include those cases in which the amplified product is an observable label that does not have specific affinity for an imager strand. In one embodiment, the undecodable amplification product could be a fluorophore, chromogenic stain, or nanoparticle.

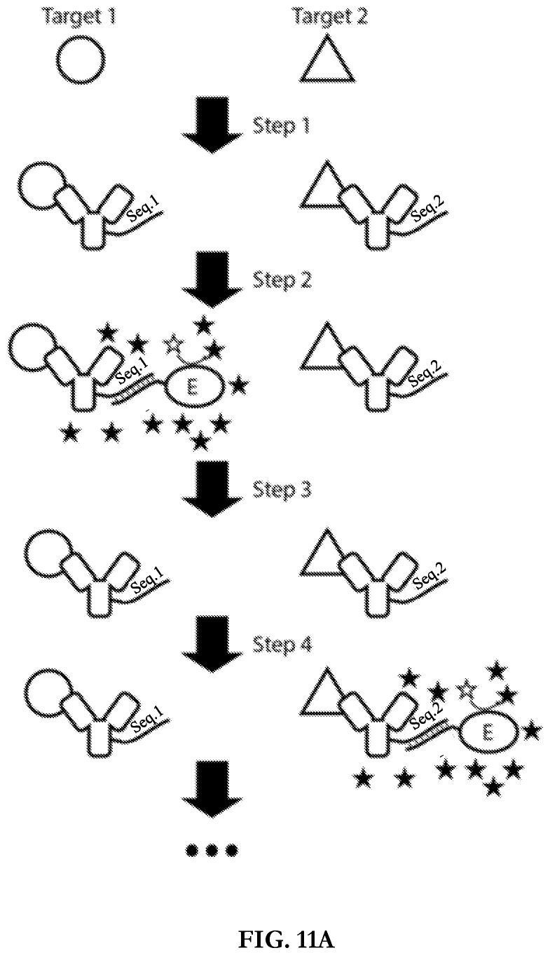

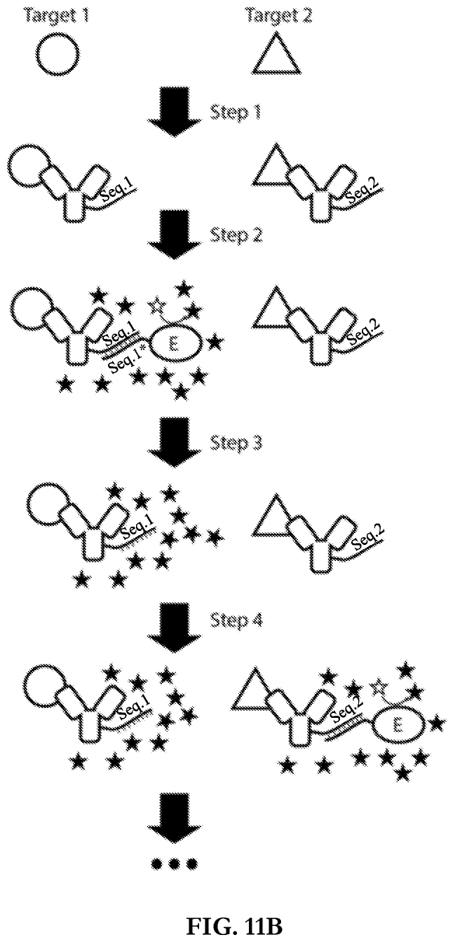

In one embodiment, a method to produce undecodable amplification products comprises: (1) contacting a sample being tested for the presence of one or more targets with one or more target-specific binding partners, wherein each target-specific binding partner is linked to a nucleic acid strand and wherein target-specific binding partners of different specificity are linked to different nucleic acid strands, (2) optionally removing unbound target-specific binding partners, (3) contacting the sample with enzyme-labeled strands capable of binding a docking strand, wherein the nucleic acid strand in (1) is either a primer strand or a docking strand and if the nucleic acid strand is a primer strand, it is linked to a docking strand, (5) optionally removing unbound enzyme-labeled strands, (4) contacting the sample with a substrate for the enzyme, (5) allowing an enzymatic reaction to produce an amplification product, (6) quenching the enzymatic reaction, (7) imaging the sample to detect the presence or absence of one or more targets, (8) removing the amplification product, and (9) repeating a subset of steps 1-9. Examples of enzymes that could be used include HRP, AP, GO, s-gal. When the enzyme is linked to an imager strand, i.e. a strand capable of binding a docking strand), sequential amplification and imaging can be carried performed (FIGS. 11A-B). FIG. 11A illustrates a method for sequential amplification and sequential imaging. Here, a method is employed to remove or inactivate the amplification product between each imaging round. Removing or inactivating the amplification product can be done by carefully choosing the substrate. For example, one may use a chromogenic substrate of HRP that is soluble in sample-friendly organic solution (e.g., 3-amino-9-ethylcarbazole, which is alcohol-soluble, PMID 19365090). In this case, after staining of docking strand-conjugated antibodies (FIG. 11A, Step 1), introducing the imager strand-conjugated HRP for one target and the substrate (FIG. 11A, Step 2), and imaging the sample, one can use alcohol (e.g., methanol) to remove the HRP product and remove the imager strand using any of the method described herein or their combination. Next one can introduce the imager strand-conjugated HRP for another target and repeat the process.

One may also use the TSA amplification method where the fluorophore can be readily bleached. For example, many cyanine fluorophores and Alexa fluorophore can be readily bleached by hydrogen peroxide in acidic or basic conditions (PMID: 26399630). Alternatively, one can synthesize a TSA dye that contains a cleavable bond between the tyramide and the fluorophore. In this case the fluorophore can be inactivated by cleaving this bond and washing.

FIG. 11B illustrates a method for sequential amplification and simultaneous imaging. In this case, after staining of docking strand-conjugated antibodies (FIG. 11B, Step 1), introducing the imager strand-conjugated enzyme for one target and the substrate (FIG. 11B, Step 2, using HRP and TSA for example) to generate amplified product, one can remove the imager strand-conjugated enzyme without removing the amplified product, and repeat multiple rounds of amplification for multiple targets prior to imaging the sample in a single imaging step.

One may also use RCA, HCR, and HDR to achieve signal amplification without decoding the amplification product. For example, after multiplexed antibody staining, one can add reagent (circular template in the case of RCA, and substrate hairpins in the case of HCR and HDR) that only supports the polymerization/dendrimerization of one subset of target and directly incorporation of fluorescent dyes in the amplification product (e.g., via fluorescent-labeled nucleotides in the case of RCA, and via fluorophore-labeled hairpin substrate in the case of HCR and HDR). After imaging of this subset of targets and inactivation of the dye by bleaching or cleavage as described above, one can introduce the reagent that supports the polymerization/dendrimerization of another subset of targets and directly incorporation of fluorescent dyes in the amplification product. This process can then be repeated.

Multiple types of signal amplification can even be used in combination. For example, Gusev et al reported combining rolling circle amplification and HRP-based signal amplification (PMID: 11438455).

One may replace the fluorophore (that is brought to the target via DNA complexes or other amplification method), by other molecule or moieties that can be directly or indirectly observed. These molecules or moieties include, but are not limited to, metal particles, plasmonic enhancers, and proteins.

b) Nonlinear Amplification

A nonlinear DNA template could be employed for signal amplification as a circular amplification strand. A circular oligo, with complementarity to a docking strand, can be generated separately from the amplification method. For example, ex situ ligation could be performed on a template DNA strand to form a circular strand of DNA. A circular strand could be hybridized to a docking strand that is attached to a target-specific binding partner before contacting the sample. Alternatively, the target-specific binding partner could first be used to stain the sample, and then subsequently the circular strand could be introduced to the sample to hybridize with the docking strand on the target-specific binding partner. Following the formation of a complex wherein a circular strand is attached to a docking strand that is linked to a target-specific binding partner, rolling circle amplification (RCA) could be carried out. This method offers certain advantages as it can be used to circumvent issues with inefficient in situ ligation steps.

In some situations, an amplifier strand may be employed. For example, in some embodiments, a method comprises (1) contacting a sample being tested for the presence of one or more targets with one or more target-specific binding partners, wherein each target-specific binding partner is linked to a nucleic acid strand and wherein target-specific binding partners of different specificity are linked to different nucleic acid strands, (2) optionally removing unbound target-specific binding partners, (3) contacting the sample with a nonlinear amplifier strand having complementarity to a nucleic acid strand, wherein the nucleic acid strand in (1) is either a primer strand or a docking strand (4) optionally removing unbound nonlinear amplifier strands, (5) amplifying the docking strand with rolling circle amplification (i.e., increasing the number of docking strands or introducing a plurality of docking strands), (6) contacting the sample with labeled imager strands capable of binding a docking strand, either directly or indirectly, (7) imaging the sample to detect bound labeled imager strands, (8) removing the bound labeled imager strands, and (9) optionally repeating steps (1)-(8), or any subset thereof.

In some embodiments, a polymerase may be used for RCA. In some instances, the labeled imager strands are linear strands. In some instances, the nonlinear amplifier strands are circular strands. In some instances, the nonlinear amplifier strands are branched strands. In some instances, the nonlinear amplifier strand becomes circular after ligation.

In some embodiments, amplification products may comprise a geometric shape, such as a triangle, quadrilateral, pentagon, hexagon, and the like.

c) Amplifying Signal Before Applying Imager Strands and Optionally Extinguishing Signal from the Bound Labeled Imager Strand

In some embodiments, a method to test a sample for the presence of one or more targets comprises (1) contacting the sample with one or more target-specific binding partners, wherein each target-specific binding partner is linked to a nucleic acid strand and wherein target-specific binding partners of different specificity are linked to different nucleic acid strands, (2) optionally removing unbound target-specific binding partners, (3) wherein the nucleic acid strand is a docking strand or a primer strand and if the nucleic acid is (a) a docking strand, increasing the number of docking strands associated with each target-specific binding partner or (b) a primer strand, associating more than one docking strand with the primer strand, (4) contacting the sample with labeled imager strands capable of binding a docking strand, either directly or indirectly, (5) optionally removing unbound labeled imager strands, (6) imaging the sample to detect bound labeled imager strands, and (7) optionally extinguishing signal from the bound labeled imager strand. In some instances, after step (4) and after optionally performing step (5) the method further comprises increasing the number of docking strands associated with each target-specific binding partner. In some embodiments, the method further comprises removing unbound labeled imager strand after the increasing the number of docking strands. Thus, in some modes, amplifying the docking strand with rolling circle amplification occurs separately from contacting the sample with labeled imager strands having complementarity to the amplified strand. By amplified strand, we mean the product of amplification (sometimes also called the amplification product or the RCA product if rolling circle amplification is employed).

In some instances, the sample is mounted to an optically transparent support. In some embodiments, the increase in the number of docking strands associated with each target-specific binding partner is achieved using an enzyme. For example, the enzyme approaches described in Section II1.4 (a) may be employed.

d) Amplifying Signal in the Presence of Imager Strands and Optionally Extinguishing Signal from the Bound Labeled Imager Strand

In some embodiments, a method to test a sample for the presence of one or more targets comprises (1) contacting the sample with one or more target-specific binding partners, wherein each target-specific binding partner is linked to a nucleic acid strand and wherein target-specific binding partners of different specificity are linked to different nucleic acid strands, (2) optionally removing unbound target-specific binding partners, (3) contacting the sample with labeled imager strands capable of binding a docking strand, either directly or indirectly, (4) wherein the nucleic acid strand is a docking strand or a primer strand and if the nucleic acid is (a) a docking strand, increasing the number of docking strands associated with each target-specific binding partner or (b) a primer strand, associating more than one docking strand with the primer strand, wherein the amplification occurs in the presence of the imager strand (5) optionally removing unbound labeled imager strands, (6) imaging the sample to detect bound labeled imager strands, and (7) optionally extinguishing signal from the bound labeled imager strand. In some instances, after step (4) and after optionally performing step (5) the method further comprises increasing the number of docking strands associated with each target-specific binding partner. In some embodiments, the method further comprises removing unbound labeled imager strand after the increasing the number of docking strands.

In some instances, the sample is mounted to an optically transparent support. In some embodiments, the increase in the number of docking strands associated with each target-specific binding partner is achieved using an enzyme. For example, the enzyme approaches described in Section II1.4 (a) may be employed.

The imager strands may have complementarity to the docking strand. The imager strand may be a circular imager strand for rolling circle amplification. The imager strand may be an imager strand that circularizes in the presence of the docking strand and ligase. In some embodiments, the imager strand may comprise at least 2, 3, 4, 5, 6, 7, 8, 9, or 10 regions that are complementary to the docking strand.

5. A Method to Test a Sample Mounted to an Optically Transparent Support

In some embodiments, a method to test a sample mounted to an optically transparent support for the presence of one or more targets comprises (1) contacting the sample with one or more target-specific binding partners, wherein each target-specific binding partner is linked to a nucleic acid strand and wherein target-specific binding partners of different specificity are linked to different nucleic acid strands, (2) optionally removing unbound target-specific binding partners, (3) wherein the nucleic acid strand is a docking strand or a primer strand and optionally if the nucleic acid is (a) a docking strand, increasing the number of docking strands associated with each target-specific binding partner or (b) a primer strand, associating more than one docking strand with the primer strand (4) contacting the sample with labeled imager strands capable of binding a docking strand, either directly or indirectly, wherein the labeled imager strands are provided in a liquid medium or buffer solution (5) optionally removing unbound labeled imager strands, (6) optionally removing liquid to create a liquid-free sample, (7) affixing a second optically-transparent material parallel to the first support, and (8) imaging the sample to detect bound labeled imager strands.

In some embodiments, the second optically-transparent material is glass or plastic. In some instances, the second optically-transparent material is from about 5 microns to 5 mm, from 50 microns to 500 microns, or from 500 microns to 5 mm from the first support. In some instances, the imaging is carried out with an upright microscope.

In some embodiments, optionally removing liquid to create a liquid-free sample comprises preparing the sample for storage, such as long-term storage for at least 4 hours, 1 day, 3 days, 1 week, 2 weeks, or one month. In some embodiments, optionally removing liquid to create a liquid-free sample increases sample handling convenience because the user does not need to keep the sample hydrated.

By optionally removing liquid to create a liquid-free sample, this means removing at least about 90%, 91%, 92%, 93%, 94%, 95%, 96%, 97%, 98%, 99%, or 100% of the liquid in a sample (namely, the liquid previously in the sample and associated with the other sample components, such as the sample itself, the docking strand, the imager strand, etc.). In some embodiments, the mounting medium comprises air. In other embodiments, the mounting media comprises a mounting media in a gel formulation. In some embodiments, the mounting media comprises a formula that begins as a liquid but changes to a gel or solid as time elapses (such as a hardening material, glue, cement, or other optically transparent and similarly-functioning material).

In other embodiments, the liquid in the sample may be replaced by a liquid mounting media such as a saline-based buffered solution (such as PBS).

Mounting media may be used to hold a sample in place, to prevent a sample from drying out, to more closely match the refractive index of the objective you will use, to prevent photobleaching (when not desired), and to preserve a sample for long-term storage. The choice of mounting media depends on the sample type, the imaging strategy, which observable moiety is used, and the objectives of the user (whether the user wishes to hydrate the sample or whether the user wishes to store the sample).

In some embodiments, a method to test a fixed sample mounted to an optically transparent support for the presence of one or more targets comprises (1) contacting the sample with one or more target-specific binding partners, wherein each target-specific binding partner is linked to a nucleic acid strand and wherein target-specific binding partners of different specificity are linked to different nucleic acid strands, (2) optionally removing unbound target-specific binding partners, (3) wherein the nucleic acid strand is a docking strand or a primer strand and if the nucleic acid is (a) a docking strand, increasing the number of docking strands associated with each target-specific binding partner or (b) a primer strand, associating more than one docking strand with the primer strand, (4) contacting the sample with labeled imager strands capable of binding a docking strand, either directly or indirectly, (5) optionally removing unbound labeled imager strands, (6) optionally removing liquid to create a liquid-free sample, (7) affixing a second optically-transparent material parallel to the first support, and (8) imaging the sample to detect bound labeled imager strands.

In the various multiple embodiments, the optically transparent support and the second optically transparent material parallel to the first support may comprise a flow cell. In the various multiple embodiments, by parallel, it includes geometrical arrangements that are perfectly parallel, as well as those that deviate from parallel by up to 1°, 2°, 3°, 4°, 5°, 6°, 7° 8°, 9° or 10°.

6. Sample Reinterrogation

In some embodiments, users may desire to reinterrogate a sample for the same target multiple times. When reinterrogation is desired, the multiplex imaging is conducted by conducting another round of imaging with the same imager stand. Thus, when a user desires to image different targets, the imager strand has a unique nucleotide sequence relative to all other labeled imager strands. When a user desires to image the same target multiple times, the repeated steps use an imager strand that does not have a unique nucleotide sequence relative to all other labeled imager strands, but instead has the same sequence as a previously employed imager strand.

Thus, in some embodiments, a method comprises (1) contacting a sample being tested for the presence of one or more targets with one or more target-specific binding partners, wherein each target-specific binding partner is linked to a nucleic acid strand and wherein target-specific binding partners of different specificity are linked to different nucleic acid strands, (2) optionally removing unbound target-specific binding partners, (3) wherein the nucleic acid strand is a docking strand or a primer strand and optionally if the nucleic acid is (a) a docking strand, increasing the number of docking strands associated with each target-specific binding partner or (b) a primer strand, associating more than one docking strand with the primer strand, (4) contacting the sample with labeled imager strands capable of binding a docking strand, either directly or indirectly, (5) optionally removing unbound labeled imager strands, (6) imaging the sample to detect presence, location, and number of bound labeled imager strands, (7) extinguishing signal from the bound labeled imager strand, and (8) repeating steps (3)-(6) or (3)-(7), with a labeled imager strand optionally having a unique nucleotide sequence relative to all other labeled imager strands.

B. Methods of Extinguishing Signal from the Bound Labeled Imager Strand

Various methods can be used to extinguish a signal from a bound labeled imager strand and this may be desired so that the same type of detectable moiety (such as a fluorophore) may be used on multiple imager strands so that the experiment is not spectrally limited.

In other words, removing the set of imager strands or inactivating the observable moieties on the imager strands allows for spectrally-unlimited multiplex imaging. Some prior methods of multiplex imaging were limited by the number of colors of fluorophores or other imaging agents available. Removing the imager strands, removing labels from imager strands, or inactivating the observable moieties allows for reuse of the same colors of fluorophores in a single experiment. In some embodiments, ideally, as much of the signal should be removed to ensure as low backgrounds as possible for continued imaging. In some embodiments, 100% of the prior signal-generating moiety is removed or destroyed, while in some embodiments at least about 90%, 91%, 92%, 93%, 94%, 95%, 96%, 97%, 98%, or 99% of the prior signal-generating moiety is removed or destroyed.

Thus, extinguishing the signal from the labeled imager strand includes any method for removing the imager strand from binding, directly or indirectly, to the docking strand, removing the label from the imager strand, or inactivating the label on the imager strand.

All methods for extinguishing the signal from imager strands may be applied to docking strands as well. In some embodiments, extinguishing the signal from the bound labeled imager strand involves disrupting the link between docking strand (or primer strand) and target-recognition moiety. In some embodiments, the docking strand comprises a photocleavable linker that can be cleaved photochemically (e.g. by UV exposure, visible light, infrared, near infrared, x-ray, microwave, radio waves, or gamma rays). In some embodiments, the docking strand (or primer strand) itself contains a moiety that can be cleaved by an enzyme. Examples of such enzymatically cleavable moieties include but are not limited to ribonucleotides, which can be cleaved by a variety of RNases; deoxyuridines, which can be cleaved by enzyme combinations such as USER (New England Biolabs); and restriction sites, which can be cleaved by sequence-specific nicking enzymes or restriction enzymes. In some embodiments, the docking nucleic acid comprises a deoxyuridine, in which the uracil group may be cleaved by uracil-DNA glycosylase. In some embodiments, the docking nucleic acid comprises an abasic site, which may be cleaved by endonuclease.

One non-limiting example of Exchange Imaging is DNA Exchange Immunofluorescence, where one uses antibodies as the target-recognizing molecules to image target proteins or other biomolecules, uses DNA oligonucleotides as docking strands, and uses DNA oligonucleotides that are complementary to the docking strands and labeled with fluorophores as the imager strands. A user may extinguish the signal from the labeled imager strand by using high temperature, denaturant, DNA helicase, DNase, and/or strand displacement, or may remove the fluorophores on the imager strands by chemical cleavage, enzymatic cleavage, chemical bleaching, photo-bleaching, and/or photochemical bleaching.

1. Nucleic Acid-Degrading Enzymes

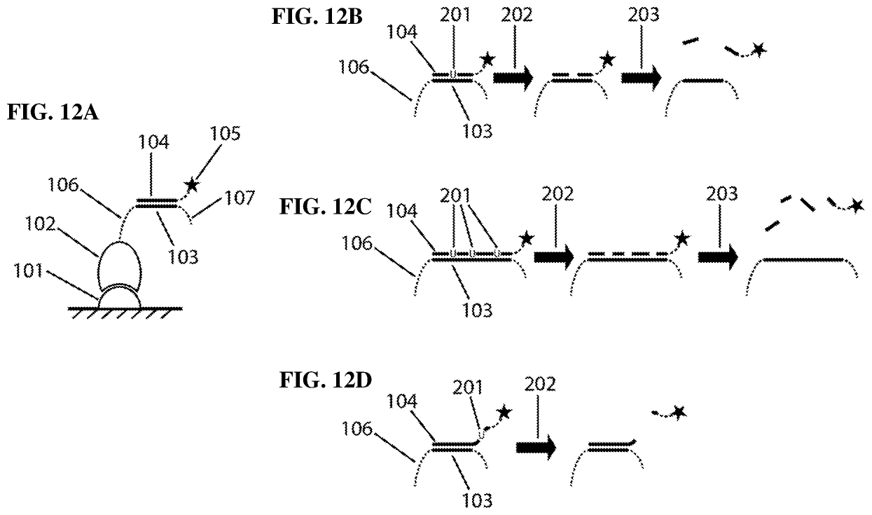

A number of enzymes can break the covalent bonds within a nucleic acid molecule. For example, some glycosylases can remove the base from the sugar moiety of a nucleotide, endonucleases can cut the bond within the phosphodiester bridge inside the nucleic acid molecule, while exonucleases can similarly break the phosphodiester bridge at the 5′ or 3′ terminal of the nucleic acid molecule in a sequential fashion. Another example comprises DNAzymes or deoxyribozymes, oligonucleotides with catalytic activity capable of cleaving the phosphodiester bond in nucleic acid molecules. All these types of enzymes may be engineered for imager strand removal (FIG. 12) and constitute enzymatically cleaving, modifying, or degrading the labeled imager strand nucleic acids.

Glycosylase. If a glycosylase can specifically remove a base that participates the base-pairing between the Docking Strand and the Imager Strand, it can reduce the strength of interaction between the two strands. For example, one can use deoxyuridine (dU) to replace deoxythymidine (dT) in the Imager Strand. dU can pair with dA in the Docking Strand just like the dT does, but can be specifically removed by Uracil-DNA Glycosylase (UDG, commercially available from New England Biolabs, Cat #M0280S). This reaction will result in abasic site(s) on the Imager Strand. Such abasic sites can be further cleaved by Endonuclease VIII. This will further promote the dissociation between the remnant of Imager Strand and the Docking Strand. Enzyme blend comprising both UDG and Endonuclease VIII is also commercially available (e.g., from New England Biolabs, under the tradename USER, Cat #M5505S). One may place from about 1 to 20, 1 to 15, 1 to 10, or 1 to 5 dU nucleotides in the Imager Strand. With USER, the dUs may be placed in a way that after removal of U, the remnants are short enough (e.g., less than or equal to about 9, 8, 7, 6, 5, 4, 3, 2, or 1 nucleotides) that they dissociate spontaneously and quickly. If only UDG (i.e., no Endonuclease VIII) is used, the removal of dU units could destabilize the strand enough to facilitate removal. Total number of base pairs between the imager strand and docking strand after dU removal may be less than or equal to 9, 8, 7, 6, 5, 4, 3, 2, or 1 nucleotides. Thus, in some embodiments the imager strand or intermediate strand may comprise at least one U capable of cleavage by USER.

The sequence of the remnants, as well as the temperature, will impact how short the remnants should be to dissociate spontaneously. For example, a sequence high in GC content might have more binding affinity at a shorter length than another sequence at a longer length. Thus, in some instances, a 9-mer may be sufficient for stable binding and in other instances a 9-mer may be sufficient to dissociate. A person of ordinary skill in the art can evaluate the sequences, temperatures, and affinities, here and in the cleavage of non-natural nucleotides discussed below.

Restriction endonuclease and nicking endonuclease. One may engineer a restriction site in the docking strand:imager strand duplex. This allows the usage of the corresponding restriction endonuclease to cut such restriction site, which breaks the linkage between the target and the signal-generating moiety of the imager strand. As an example, Cas9 (CRISPR associated protein 9) is an RNA-guided endonuclease that can be used to specifically cleave docking: imager strand duplexes, by engineering a specific recognition site in the corresponding sequences. This results in both strands being cleaved, preventing one from re-interrogating the corresponding target. To solve this problem, one can use nicking endonuclease which only cut one strand. As an example, Cas9 nickases are Cas9 enzymes that have been engineered to only include one active cleaving site, leading to single strand cuts, while conserving the high specificity of Cas9. One can design the restriction site in a way that only the imager strand is cut, and that the remnant of imager strand that carries the signal-generating moiety is sufficiently short (e.g., <7 nucleotide) that it dissociates spontaneously and quickly from the docking strand. Other examples of endonucleases with site specific activity include but are not limited to: zinc finger nucleases, transcription activator-like effector nucleases (TALENs), and deoxyribozymes.

RNase. One may make some or all of nucleotides in the imager strand RNA nucleotides (also called ribonucleotides), instead of DNA nucleotides (also called deoxynucleotide). Such RNA nucleotides can be removed by RNase. If the docking strand is comprised of DNA nucleotides and the imager strand contains RNA nucleotides, such RNA nucleotides in the DNA:RNA heteroduplex can be removed by RNase H.

Polymerase. The imager strand can also be removed by using polymerases with strand-displacement activity or 5′-to-3′ exonuclease activity. For example, one can engineer a hairpin structure at the 3′ end of the docking strand made of DNA. When a DNA polymerase with strand displacement activity (e.g., phi29, Bst, Vent) is introduced and supplied with suitable buffer and dNTPs, the 3′ of the docking strand can be extended, during which the imager strand is displaced (FIG. 13C). The self-priming hairpin can also be engineered on the imager strand (FIG. 18a-b), for which the signal-general moiety can be either attached to the imager strand directly (FIG. 13A), or attached to the imager strand via DNA hybridization (FIG. 13B). When a DNA polymerase with 5′-to-3′ exonuclease activity (e.g., DNA polymerase I, Taq) is introduced and supplied with suitable buffer and dNTPs, and a self-priming hairpin is engineered at the 3′ end of the docking strand, the 3′ can be extended, during which the imager strand is degraded (FIG. 13E). Similar effect can be achieved if the self-priming hairpin is engineered at the 3′ end of the imager strand (FIG. 13D). Note that the self-priming hairpin can also be replaced by a stable duplex (e.g., FIG. 13F).

Cleavage of non-natural nucleotides. Non-natural nucleotide that serve as substrates for particular enzymes may be used. For example, 8-oxoguanine may be cleaved by DNA glycosylase OGG1. Abasic sites may also be incorporated into a DNA strand, such as an imager strand, which may be cleaved by an endonuclease. For example, a 1′,2′-Dideoxyribose, dSpacer, apurinic/apyrimidinic, tetrahydrofuran, or abasic furan may be cleaved by Endonuclease VIII. Thus, in some embodiments the imager strand or intermediate strand may comprise at least one abasic site capable of cleavage by Endonuclease VIII. In some embodiments the imager strand or intermediate strand may comprise at least one deoxyuridine and at least one abasic site capable of cleavage by USER, UDG, or Endonuclease VIII. Photocleavable spacers or RNA abasic sites may also be used, such as ribospacer (rSpacer) or Abasic II modification. Other pairs of non-natural nucleotides and their paired enzymes may be employed.

Thus in some embodiments, a composition comprises (1) a label, (2) a first nucleic acid domain, a second nucleic acid domain, and a third nucleic acid domain, wherein each nucleic acid domain is from about 1 to 9 nucleotides long (for example, about 9, 8, 7, 6, 5, 4, 3, 2, or 1 nucleotides), (3) a first linking moiety linking the first nucleic acid domain and the second nucleic acid domain and (4) a second linking moiety linking the second nucleic acid domain and the third nucleic acid domain, wherein both linking moieties are independently chosen from (a) an abasic site with an intact phosphodiester backbone, (b) a linker cleavable by a nucleic acid glycosylase, or (c) a restriction site or nicking site. In some embodiments, additional nucleic acid domains are linked by additional linking moieties. In some embodiments, at least one linking moiety is an abasic site (apyrimidinic) with an intact phosphodiester backbone. In some embodiments, at least one linking moiety is susceptible to cleavage from Endonuclease VIII. In some embodiments, the nucleic acid domains comprise DNA and in some the nucleic acid domains comprise RNA. In some aspects, at least one linking moiety comprises at least one non-natural nucleotide. In some aspects, at least one linking moiety comprises 8-oxoguanine.

In some embodiments, methods of removing imager strand may be combined with amplification steps. In some embodiments, a method to test a sample for the presence of one or more targets comprises (1) contacting the sample with one or more target-specific binding partners, wherein each target-specific binding partner is linked to a nucleic acid strand, directly or indirectly and wherein target-specific binding partners of different specificity are linked to different nucleic acid strands, (2) optionally removing unbound target-specific binding partners, (3) wherein the nucleic acid strand is a docking strand or a primer strand and if the nucleic acid is (a) a docking strand, increasing the number of docking strands associated with each target-specific binding partner or (b) a primer strand, associating more than one docking strand with the primer strand, (4) contacting the sample with labeled imager strands capable of binding a docking strand, directly or indirectly, wherein the labeled imager strands comprise the composition described immediately above, (5) optionally removing unbound labeled imager strands, (6) imaging the sample to detect bound labeled imager strands, (7) removing the bound labeled imager strands from the docking strands, wherein the labeled imager strands are removed from the docking strands by enzymatically cleaving, modifying, or degrading the labeled imager nucleic acids, and (8) optionally repeating steps (1)-(7), or any subset thereof.

In some aspects, the labeled imager nucleic acids are removed by enzymatically cleaving the labeled imager strand.

C. Description of Samples

1. Types of Samples

Various types of samples may be imaged using these methods. In some embodiments, the sample is a fixed sample. In some embodiments, the sample is a cell, cell lysate, tissue, tissue lysate, and or a whole organism. In some embodiments, the sample is a cell or tissue sample, a cell or tissue lysate, or a bodily fluid. In some embodiments, the sample is tissue and the imaging comprises in-tissue multiplexing for immunostaining.

The sample may be provided in a liquid medium or buffer solution.

2. Antigen Retrieval

In some embodiments, staining a sample with a target-specific binding partner requires specific conditions and not all target-specific binding partners will bind to their antigens under the same conditions. This may be because their target antigens are not available under the same conditions.

Thus in some embodiments, a method to test a sample for the presence of one or more targets further comprises exposing a different set of previously unavailable targets; using one or more different target-specific binding partners, and using a labeled imager strand having a unique nucleotide sequence relative to at least one other labeled imager strand.

3. Description of Targets and Use in Identifying Biomarkers

In some embodiments, the method is useful for identifying a biomarker. In some instances, samples are imaged and data analysis performed on those samples. In some embodiments, multiple targets are tested for using corresponding target-specific binding partners for each target. In some instances, the relationship between different targets may be assessed; for example, a user might seek to determine the relationship of multiple markers to a disease state and conclude that the disease sample has increased levels of A, decreased levels of B, and levels of C within a certain range, as compared to healthy tissue that does not have that biomarker distribution.

In some embodiments, at least 10, 96, 100, 384, or 500 samples are imaged and data analysis performed on those samples.

In some embodiments, at least 5, 10, 15, 25, 30, 50, 75, or 100 or more targets are tested for using corresponding target-specific binding partners for each target.

D. Equipment and Software

1. Imaging Chamber

In some embodiments, an imaging chamber can be employed. In some instances, an imaging chamber is a fixed chamber with no inlet and no outlet. In some embodiments, an imaging chamber has a single inlet/outlet combination. In other instances, an imaging chamber allows for flow and is designated a flow cell. A flow cell may be comprised of a first optically transparent support in combination with a second optically transparent material (such as a glass or plastic coverslip) to provide a flow cell with a top and bottom surface and fluid flow between them. If a first and second optically transparent material are used, they may be placed parallel to each other. By parallel, it includes geometrical arrangements that are perfectly parallel, as well as those that deviate from parallel by up to 1°, 2°, 3°, 4°, 5°, 6°, 7° 8°, 9°, or 10°. In some embodiments, the second optically transparent material is in close proximity to the first optically transparent material, such as about 5 microns to 5 mm, from 50 microns to 500 microns, or from 500 microns to 5 mm.

An imaging chamber may also be comprised of a first optically transparent support and a gasket (also referred to as an isolator or spacer). The gasket may be open to the air on the top surface or it may be closed and have an optically transparent top surface. The gasket may have a combined inlet/outlet or it may have both an inlet and an outlet. The gasket may also have no outlet. The gasket may be plastic, rubber, adhesive. A gasket may comprise a CoverWell Chamber Gasket (Thermo Fisher), an ultra-thin sealed chamber for upright and inverted microscopes (Bioscience Tools), or an incubation chamber (Grace Bio-Labs, including HybriSip™ hybridization covers, HybriWell™ sealing system, CoverWell™ incubation chambers, imaging spacers, SecureSeal™ hybridization chambers, FlexWell™ incubation chambers, FastWells™ reagent barriers, and Silicone Isolators™).

In some instances, a gasket may be employed along with a coverslip forming the top surface of an imaging chamber or flow cell.

Imaging chambers, such as but not limited to flow cells, may be reusable or disposable.

E. Kits

In some embodiments, a kit for detecting Nₘ targets with N_{ch} labels used in the kit wherein Nₘ is greater than N_{ch}, comprises: 1) target-specific binding partners linked to nucleic acid strands, wherein target-specific binding partners of different specificity are linked to different nucleic acid strands; (2) labeled imager strands comprising i) multiple labeled imager strands capable of binding the same nucleic acid strand associated with the target-specific binding partner, to either the same or different domains within the nucleic acid strand, wherein the multiple imager strands comprise a different type of label and/or (ii) at least one labeled imager strand capable of binding the same nucleic acid strand associated with the target-specific binding partner, wherein the imager strand comprises more than one type of label; and (3) optional buffers, amplification reagents, and/or reagents to remove bound imager strands.

### EXAMPLES

### Example 1. 3-Channel, 6-Plex, Simulation

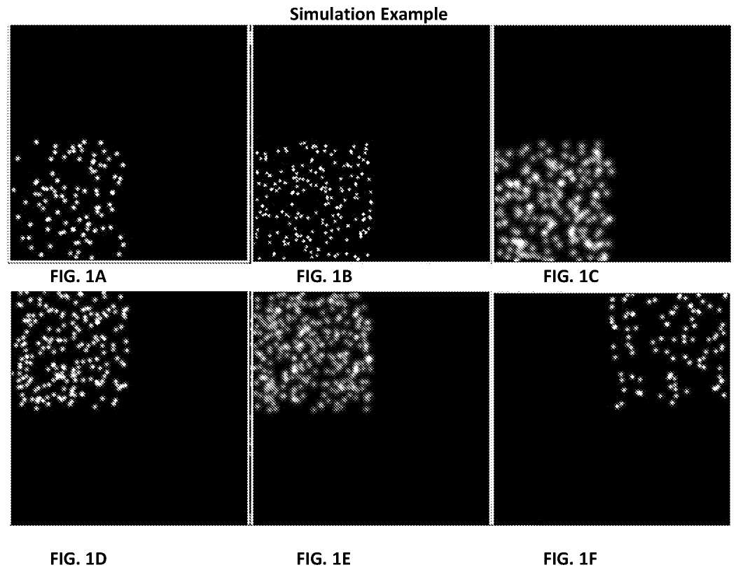

A set of model images for six different targets were created and they were encoded into a synthetic image with three spectral channels. For the model images of each target (FIGS. 1A-1F), a collection of Gaussian spots in slightly different sizes were used to simulate individual cells and the spots were confined to different quadrants of a 500×500 pixel image in order to prevent the signals from different basis sets occurring in the same pixel. The choice of using quadrants is simply a programming convenience; any other pattern, regular or random, may be used as long as the simulated signals of each basis set are spatially separated.

As shown in FIGS. 1A-1F, for model image of targets A, B and C, the spots were confined to the lower left quadrant; for the model image of targets D and E, the upper left, and for the model image of target F, the upper right. This ensures that targets in the different groups do not occur in the same pixel (and thus meeting the rule that the basis sets are mutually exclusive groups of targets).

These model images and their dual color intensity ratios (Table 12) were used to produce the simulation of the measured images.

TABLE 12
Encoding scheme for simulated images

| | Basis | Channel 1 | Channel 2 | Channel 3 |
|---|---|---|---|---|
| Targets | set | ratio | ratio | ratio |
| A | 1 | 0.5 | 0.5 | 0.0 |
| B | 1 | 0.5 | 0.0 | 0.5 |
| C | 1 | 1.0 | 0.0 | 0.0 |
| D | 2 | 0.0 | 0.5 | 0.5 |
| E | 2 | 0.0 | 1.0 | 0.0 |
| F | 3 | 0.0 | 0.0 | 1.0 |

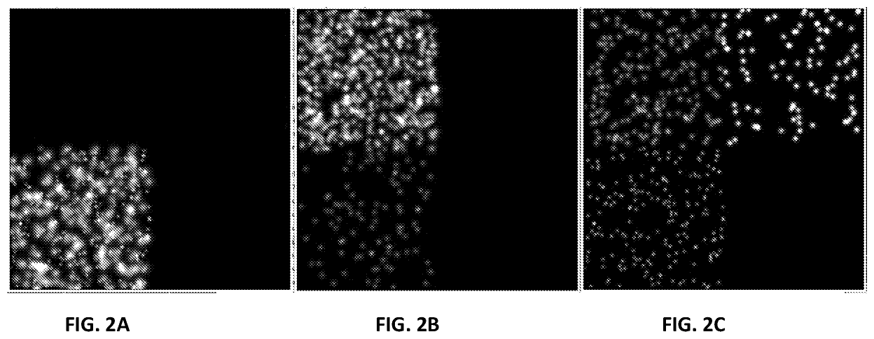

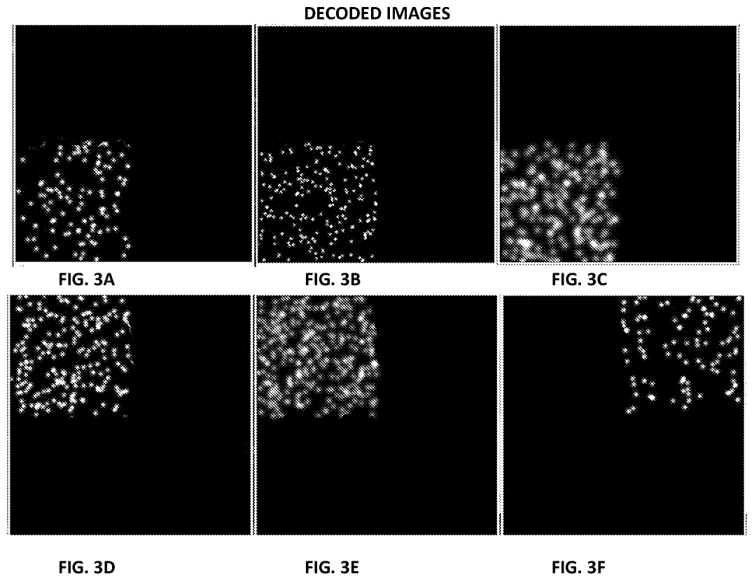

FIGS. 2A-2C show the resulting encoded images of each spectral channel corresponding to each label 1-3. The images of FIGS. 2A-2C were used as input image for the decoding method described above and the encoding scheme in Table 12, and the output, the decoded images of the six targets, is shown in FIGS. 3A-3F, essentially identical to the input images shown in FIGS. 1A-1F.

### Example 2. 4-Channel, 6-Plex Imaging with Dual-Color Labeling Agent Comprising Multiple Labeled Imager Strands with Different Labels

Sample Preparation

Formalin-fixed, paraffin-embedded human tonsil tissue was baked at 60° C. for 30 minutes. The tissue was de-paraffinized using a Gemini Automated Slide Stainer (Thermo Fisher) by incubating 3 times in Xylene for 10 minutes per each incubation followed by 2-minute incubations in 99% ethanol, 99% ethanol, 90% ethanol, 70% ethanol, 50% ethanol, and deionized water. Antigen retrieval was performed on the slides using a Lab Vision™ PT Module (Thermo Fisher) at pH 9.0 for 20 minutes at 100° C. The tissue was rinsed once in phosphate buffered saline (PBS) to remove any remaining buffer and paraffin, and then was sectioned with a hydrophobic marker.

The tissue was blocked with 100 L of Blocking Solution (Ultivue) and incubated for 1.5 hours at room temperature. After the blocking solution was removed, the tissue was contacted with 100 L of Antibody Diluent (Ultivue) containing docking strand-attached target-specific binding partners (anti-CD3-(docking strand 1), anti-cytokeratin-(docking strand 2), anti-PDL1-(docking strand 3), anti-PD1-(docking strand 4), anti-Ki67-(docking strand 5), and anti-CD8-(docking strand 6). Then tissue was incubated for 1 hour at room temperature. The tissue was washed by submerging in PBS three times.

Then, the tissue was provided with 100 L of Preamplification Solution (Ultivue), incubated for 25 minutes at room temperature, and washed three times with PBS. Next, the tissue was provided with 100 L of Amplification Solution (Ultivue), incubated for 2 hours at 30° C., and washed three times with PBS. Then, the tissue was stained with a nuclear counterstain, incubated for 15 minutes at room temperature, and washed three times with PBS.

Next, the tissue was provided with 100 L of Probe Solution (Ultivue) containing (imager strand 1)-TRITCfluor, (imager strand 1)-Cy5fluor, (imager strand 2)-Cy7fluor, (imager strand 2)-Cy5fluor, (imager strand 3)-FITCfluor, (imager strand 4)-Cy7fluor, (imager strand 5)-Cy5fluor, (imager strand 6)-TRITCfluor (for TRITC, Alexa Fluor 555 (Thermo Fisher) and for FITC, Alexa Fluor 488 (Thermo Fischer) are used), incubated for 25 minutes at room temperature, and washed three times with PBS.

The tissue was mounted and coverslipped with Prolong Antifade Diamond mounting medium (Thermo Fisher) and was stored overnight in drawer at room temperature. As a result of the staining procedure above, the following encoding scheme is prepared as shown in Table 13.

TABLE 13
Encoding scheme for Example 2

| | Basis | Label (Spectral Channel) | | | |
|---|---|---|---|---|---|
| Target | Set | Cy5 | TRITC | Cy7 | FITC |
| CD3 | 1 | X | X | | |
| CD8 | 1 | X | | X | |
| PD1 | 1 | X | | | |
| Ki67 | 2 | | X | | |
| PDL1 | 3 | | | X | |
| CK | 4 | | | | X |

Imaging

The tissue was imaged on a Zeiss Axios Scan.Z1 with the whole slide scan using the following exposure times for the 5 spectral channels: DAPI (7.61 ms); Cy5 (4.91 ms); TRITC (1.26 ms); Cy7 (5.81 ms); FITC (10.71 ms).

Consecutive sections of the same tissue sample were imaged in the following configurations:
1. Full encoding (all six targets)
2. Single-color labeled targets alone (PD1, Ki67, PDL1, CK)
3. Dual-color labeled target CD3 alone
4. Dual-color labeled target CD8 alone
5. No targets

The images obtained in steps 3 and 4 were used to determine the relative brightness of the dual-color labeled targets in each of their two spectral channels. These images were used for comparison with the full encoding results (from step 1) by constructing an average of the two channels. The images collected of the single-color labeled targets in step 2 were used for comparison with the full encoding results (from step 1). The images collected in step 5 was used to assess background autofluorescence.

Decoding

From the images of the dual-color labeled targets alone (imaging configurations 3&4 above) and the image processing method described in paragraph [0088] above, the relative brightness of the dual color reagents for CD3 was found to be 0.55 (TRITC/Cy5) and for CD8 0.626 (Cy7/Cy5).

To build the decoding table for CD3 (Table 14), we found the relative brightness of the Cy5 channel to be 1/(1+0.55)=0.645 and therefore the TRITC channel relative brightness is 1-0.645=0.355. For CD8 we found the relative brightness of Cy5 to be 1/(1+0.626)=0.615 and therefore the Cy7 relative brightness is 1-0.615=0.385. All other relative brightness values for these markers was 0.0 since they are dual color only. The table entries for PD1, Ki67, PDL1 and CK were set to 1.0 for the Cy5, TRITC, Cy7 and FITC channels respectively, 0.0 elsewhere because these are single color reagents. The resulting decoding input table is then:

TABLE 14
Decoding input table for Example 2

| | Ratios of label | | | |
|---|---|---|---|---|
| | (relative intensity | | | |
| | in spectral channel) | | | |
| Target | Cy5 | TRITC | Cy7 | FITC |
| CD3 | 0.645 | 0.355 | 0.0 | 0.0 |
| CD8 | 0.615 | 0.0 | 0.385 | 0.0 |
| PD1 | 1.0 | 0.0 | 0.0 | 0.0 |
| Ki67 | 0.0 | 1.0 | 0.0 | 0.0 |
| PDL1 | 0.0 | 0.0 | 1.0 | 0.0 |
| CK | 0.0 | 0.0 | 0.0 | 1.0 |

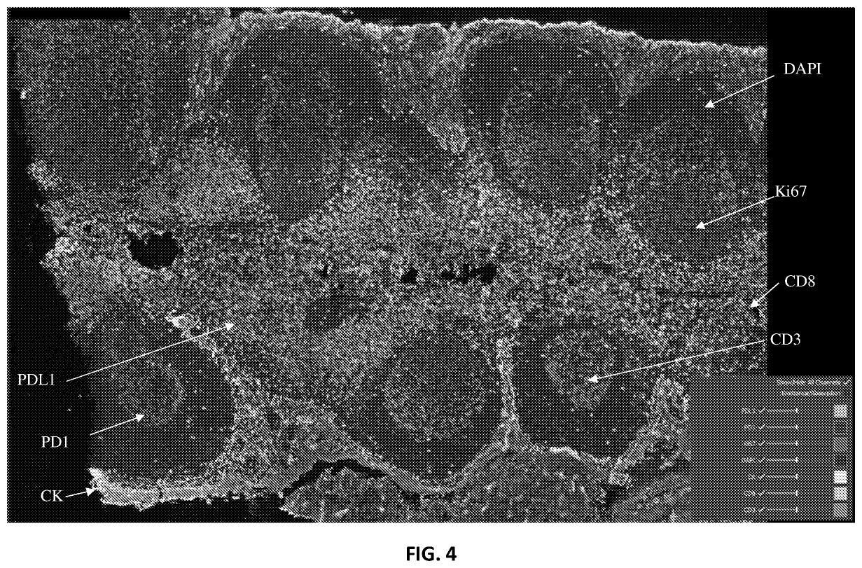

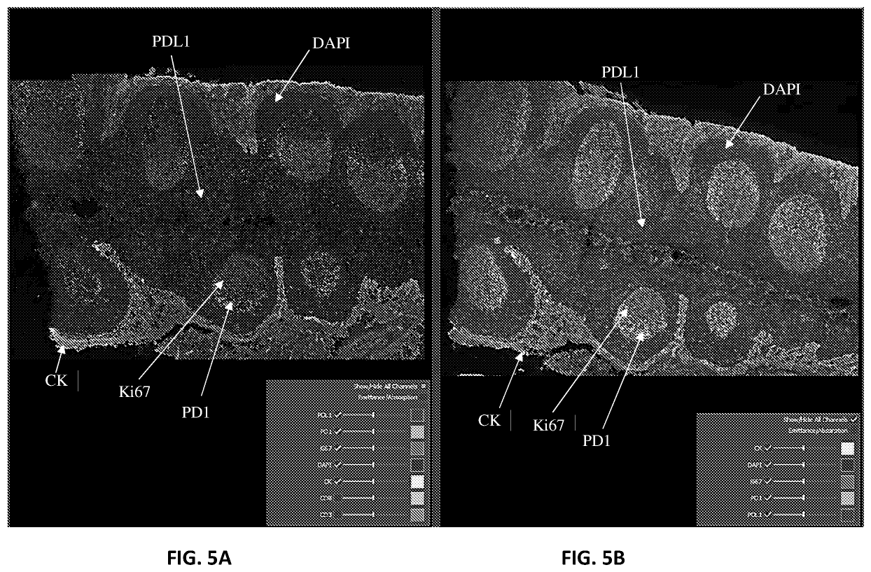

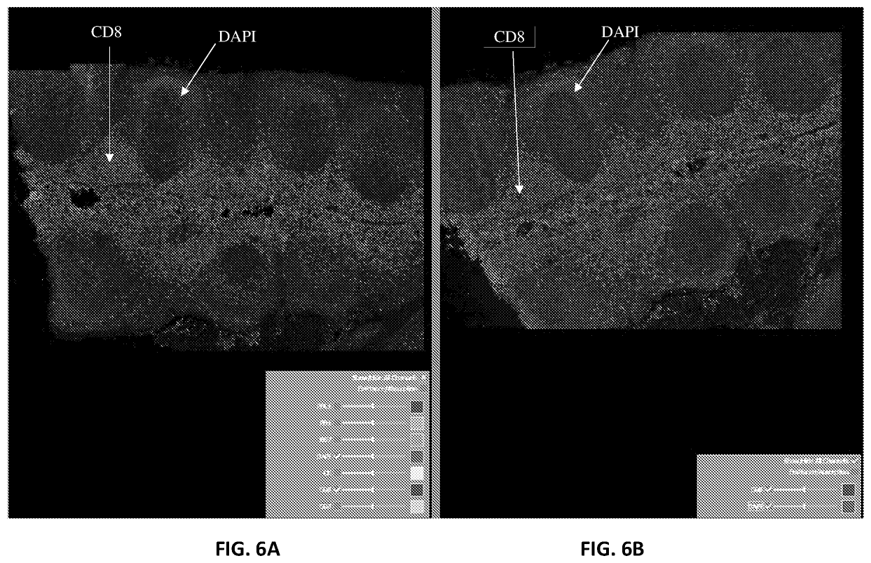

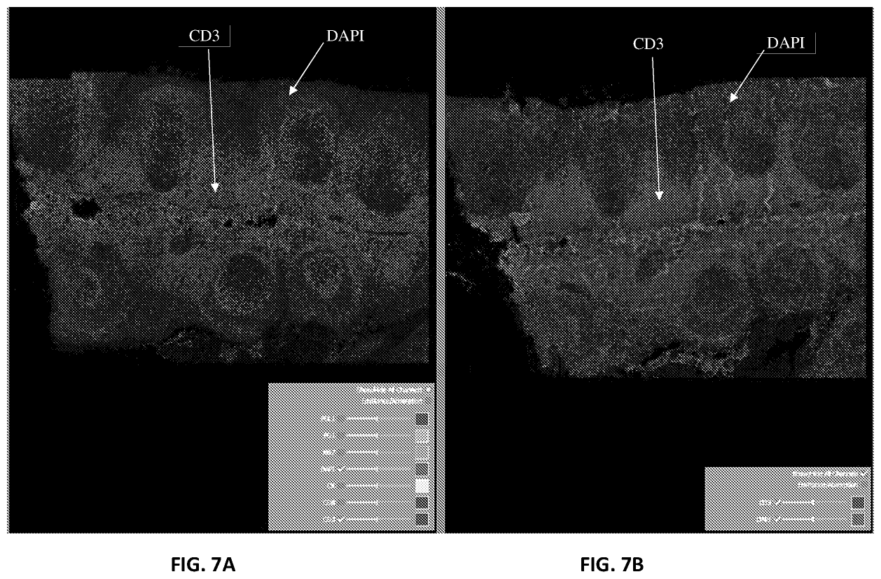

The encoded images were processed according to the method described above and the images were overlaid with each other and visualized in different pseudo colors for comparison as shown in FIG. 4. All of the 6 targets were decoded from 4 spectral channels, overlaid with the DAPI image. As shown in FIG. 5A, the staining pattern of the decoded image of monochromatic targets (PDL1, PD1, Ki67, and Cl) were compared with the images obtained from the consecutive section staining with each of the monochromatic targets (FIG. 5B). As shown in FIG. 6A, the decoded CD8 signal recovered from the full decoding of all 6 targets were compared with a consecutive section with stained with the CD8 dual color reagent alone (average of the Cy5 and TRITC channels) (FIG. 6B). As shown in FIG. 7A, the decoded CD3 signal recovered from the full decoding of all 6 targets were compared with a consecutive section with stained with the CD3 dual color reagent alone (average of the Cy5 and Cy7 channels) (FIG. 7B).

Each of the results from the full encoding of all six targets successfully showed the signals of all six targets in good agreement with the signals seen in the images obtained from the consecutive section staining (control).

### Example 3. Additional Embodiments

The following numbered items provide additional support for and descriptions of the embodiments herein.

Item 1. A multicolor multiplex imaging method comprising
(1) contacting a sample being tested for the presence of one or more targets with one or more target-specific binding partners, wherein each target-specific binding partner is linked to a nucleic acid strand and wherein target-specific binding partners of different specificity are linked to different nucleic acid strands;
(2) optionally removing unbound target-specific binding partners;
(3) contacting the sample with labeled imager strands, wherein in at least one occurrence of this step the labeled imager strands comprise (i) multiple labeled imager strands capable of binding the same nucleic acid strand associated with the target-specific binding partner, and to either the same or different domains within the nucleic acid strand, wherein the multiple imager strands comprise a different type of label and/or (ii) at least one labeled imager strand capable of binding the same nucleic acid strand associated with the target-specific binding partner, wherein the imager strand comprises more than one type of label;
(4) optionally removing unbound labeled imager strands;
(5) imaging the sample to detect bound labeled imager strands;
(6) optionally removing the bound labeled imager strands from the nucleic acid strands; and
(7) optionally repeating steps (1)-(6), or any subset thereof
   wherein imaging the sample to detect the bound labeled imager strands includes detecting Nₘ targets with N_{ch} labels used in the method wherein Nₘ is larger than N_{ch}.

Item 2. The method of item 1, wherein at least one of the labeled imager strand(s) of step (3)(i) and/or (ii) are capable of binding the same nucleic acid strand associated with the target-specific binding partner directly.

Item 3. The method of any one of items 1-2, wherein at least one of the labeled imager strand(s) of step (3)(i) and/or (ii) are capable of binding the same nucleic acid strand associated with the target-specific binding partner indirectly.

Item 4. The method of any one of items 1-3, wherein labeled imager strands of step (3)(i) and/or (ii) capable of binding the same nucleic acid strand associated with the target-specific binding partner bind to the same domain within the nucleic acid strand.

Item 5. The method of any one of items 1-3, wherein labeled imager strands of step (3)(i) and/or (ii) capable of binding the same nucleic acid strand associated with the target-specific binding partner bind to a different domain within the nucleic acid strand.

Item 6. The method of any one of items 1-5, wherein the method amplifies at least one signal.

Item 7. The method of any one of items 1-6, wherein the method does not amplify at least one signal.

Item 8. The method of any one of items 1-7, wherein Nₘ is an integer of 3, 4, 5, 6, 7, 8, 9, 10, 11, 12, 13, 14, or 15, and N_{ch} is an integer of 2, 3, 4, 5, or 6 smaller than N_{ch}.

Item 9. The method of any one of items 1-8, wherein the method results in at least one target being labeled with at least two different types of labels.

Item 10. The method of any one of items 1-9, wherein the multiple labeled imager strands capable of binding the same nucleic acid strand associated with the target-specific binding partner comprise an identical nucleotide sequence.

Item 11. The method of any one of items 1-10, wherein the nucleic acid strand is a docking strand, and the multiple labeled imager strands comprise at least two labeled imager strands capable of binding the same docking strand, directly or indirectly, but comprising a different type of label.

Item 12. The method of item 11, wherein the multiple labeled imager strands comprise at least three labeled imager strands capable of binding the same docking strand, directly or indirectly, but comprising a different type of label.

Item 13. The method of any one of items 11-12, wherein the multiple labeled imager strands are capable of binding the same docking strand, but comprise a different type of label and are provided in equal amounts.

Item 14. The method of any one of items 11-12, wherein the multiple labeled imager strands are capable of binding the same docking strand, but comprise a different type of label and are provided in unequal amounts.

Item 15. The method of any one of items 11-14, wherein the multiple labeled imager strands comprise a first imager strand with a first label attached and a second imager strand with a second label attached, and both the first and second imager strands have a nucleotide sequence complementary to the same domain of the docking strand.

Item 16. The method of any one of items 1-14, wherein the nucleic acid strand is a docking strand, and the docking strand has more than one domain of nucleotide sequence having complementarity to the labeled imager strand.

Item 17. The method of item 16, wherein the multiple labeled imager strands comprise a first imager strand with a first label attached, capable of binding to a first domain of the docking strand and a second imager strand with a second label attached, capable of binding to a second domain of the docking strand, the second domain having a nucleotide sequence different from the first domain.

Item 18. The method of any one of items 1-9, wherein at least one of the at least one labeled imager strand comprises more than one type of label.

Item 19. The method of item 18, wherein the more than one type of label comprises at least two types of labels.

Item 20. The method of item 19, wherein the more than one type of label comprises at least three types of labels.

Item 21. The method of any one of items 1-20, further comprising increasing the number of the nucleic acid strands capable of binding the labeled imager strands.

Item 22. The method of any one of items 1-10 and 18-21, wherein the nucleic acid strand linked with the target-specific binding partner is a primer strand, and the method further comprises contacting the sample with a template strand for rolling circle amplification having complementarity to the primer strand; extending the primer strand along the template strand by rolling circle amplification to produce an amplified strand including a plurality of docking strands; and contacting the sample with the labeled imager strands having complementarity to the docking strands.

Item 23. The method of any one of items 1-21, wherein the nucleic acid strand associated with the target-specific binding partner is a docking strand, and the method further comprises contacting the sample with a nonlinear amplifier strand having complementarity to the docking strand; extending the docking strand along the nonlinear amplifier strand by rolling circle amplification to produce amplified docking strands; and contacting the sample with the labeled imager strands having complementarity to the amplified docking strands.

Item 24. The method of any one of items 1-23, wherein the number of targets being detected, Nₘ, given the number of labels, N_{ch}, is represented by the following formula (1):

N_{ch}<Nₘ≤N_{ch}*(N_{ch}+1)/2 (1)

where Nₘ is an integer and N_{ch} is an integer chosen from 2, 3, 4, 5, and 6.

Item 25. The method of item 24, wherein the number of targets being detected, Nₘ, given the number of labels, N_{ch}, is represented by the following formula (2):

Nₘ=N_{ch}*(N_{ch}+1)/2 (2)

where Nₘ is an integer and N_{ch} is an integer chosen from 2, 3, 4, 5, and 6.

Item 26. The method of any of items 1-25, wherein the imaging the sample to detect the labeled imager strands further comprises obtaining fluorescent spectral data from at least one image where each pixel contains the measured intensity in N_{ch} spectral channels for corresponding N_{ch} labels; and decoding the image to provide decoded images of the Nₘ targets.

Item 27. The method of item 26, wherein the decoding is conducted by processing the fluorescent spectral data with N_{ch} spectral channels pixel-by-pixel by performing the following:
(1) determining relative intensities in each spectral channel of a label or labels for each target;
(2) based on the determined relative intensities, grouping the targets into N_{ch}, mutually exclusive basis sets, the targets being the members of each basis set commonly having non-zero intensity in one of N_{ch} channels;
(3) given the relative spectral intensity of the members of each basis set, and given the measured intensity of the sample in each pixel in each channel, adjusting the levels of member of each basis set to produce the least error in matching the basis set with the measured intensities; and
(4) selecting the basis set with the least error and assigning each element of an output array with Nₘ values to the levels determined for the members of the selected set or to zero for members not of the selected set.

Item 28. The method of item 27, wherein steps (3)-(4) are repeated for a portion of pixels of the input image.

Item 29. The method of item 27, wherein steps (3)-(4) are repeated for all of pixels in the input image.

Item 30. The method of any one of items 27-29, wherein steps (3)-(4) are conducted in parallel for multiple pixels of the input image.

Item 31. The method of any one of items 27-30, wherein the relative intensities in each spectral channel for a label or labels in step (1) are determined by capturing an image of a calibration sample containing multiple labeled imager strands for the same target, and obtaining the relative intensities in spectral channels; repeating the same for the multiple labeled imager strands associated with different targets.

Item 32. The method of any one of items 27-31, wherein the relative intensities in each spectral channel of a label or labels for each target in step (1) are determined to meet the following:
1) for each target, the sum of the relative intensities in each N_{ch} spectral channels is 1;
2) when the target is associated with only one type of label N_{ch}, the relative intensity in corresponding spectral channel N_{ch} is 1.0 and the relative intensities of the other channels are 0;
3) when the target is associated with more than one type of label, the relative intensities of each of the two spectral channels are non-zero values that sums up to 1.0, and the relative intensities of the other channels are 0; and
4) for each basis set N_{ch}, where N_{ch} is the channel number, all of the labeled imager strands include a common type of label having emission in channel N_{ch}.

Item 33. The method of any one of items 27-32, wherein the R values that represent the relative intensities of the more than one label is determined by:
(1) obtaining a calibration image of a sample which has been stained with just the multiple imager strands with two different types of labels alone and imaged under the same conditions as will be used for subsequent experiments;
(2) measuring the intensities from each spectral channel;
(3) dividing the image of one channel of the calibration image by the other to find a ratio image;
(4) creating a mask image that selects pixels in the calibration image wherever the intensity in the two channels is above a simple threshold (e.g. the threshold might be 20% of the maximum brightness for each);
(5) calculating the mean or median value, μ, of the ratio image at substantially all pixels in the mask image; and
(6) calculating the relative intensities, R₁ and R₂, in the two channels having the following relationships:

R₁/R₂=μ

R₁+R₂=1

Item 34. A kit for detecting Nₘ targets with N_{ch} labels provided in the kit wherein Nₘ is larger than N_{ch}, comprising:
1) target-specific binding partners linked to nucleic acid strands, wherein target-specific binding partners of different specificity are linked to different nucleic acid strands;
2) labeled imager strands comprising (i) multiple labeled imager strands capable of binding the same nucleic acid strand associated with the target-specific binding partner, to either the same or different domains within the nucleic acid strand, wherein the multiple imager strands comprise a different type of label and/or (ii) at least one labeled imager strand capable of binding the same nucleic acid strand associated with the target-specific binding partner, wherein the imager strand comprises more than one type of label; and
3) optional buffers, amplification reagents, and/or reagents to remove bound imager strands.

Item 35. A system for detecting a plurality of targets from fluorescence spectral data, wherein the number of targets to be detected, Nₘ, given the number of labels, N_{ch}, is represented by the following formula:

N_{ch}<Nₘ≤N_{ch}*(N_{ch}+1)/2,
where Nₘ and N_{ch} are an integer, the system comprising a fluorescent microscope, a light source, a detector, a computer processor operably connected with the detector; and a tangible non-transitory storage medium having computer-readable instructions embedded therein which, when loaded onto the computer processor, cause the processor to conduct the following:
(1) determining relative intensities in each spectral channel of a label or labels for each target;
(2) based on the determined relative intensities, grouping the targets into N_{ch}, mutually exclusive basis sets, the targets being the members of each basis set commonly having non-zero intensity in one of N_{ch} channels;
(3) given the relative spectral intensity of the members of each basis set, and given the measured intensity of the sample in each pixel in each channel, adjusting the levels of member of each basis set to produce the least error in matching the basis set with the measured intensities; and
(4) selecting the basis set with the least error and assigning each element of an output array with Nₘ values to the levels determined for the members of the selected set or to zero for members not of the selected set.

Item 36. The method of item 35, wherein steps (3)-(4) are repeated for a portion of pixels of the input image.

Item 37. The method of item 35, wherein steps (3)-(4) are repeated for all of pixels in the input image.

Item 38. The method of any of items 35-37, wherein steps (3)-(4) are conducted in parallel for multiple pixels of the input image.

Item 39. The method of any of items 35-38, wherein the relative intensities in each spectral channel for a label or labels in step (1) are determined by capturing an image of a calibration sample containing multiple labeled imager strands for the same target, and obtaining the relative intensities in spectral channels; repeating the same for the multiple labeled imager strands associated with different targets.

Item 40. The method of any of items 35-39, wherein the relative intensities in each spectral channel of a label or labels for each target in step (1) are determined to meet the following:
1) for each target, the sum of the relative intensities in each N_{ch} spectral channels is 1;
2) when the target is associated with only one type of label N_{ch}, the relative intensity in corresponding spectral channel N_{ch} is 1.0 and the relative intensities of the other channels are 0;
3) when the target is associated with more than one type of label, the relative intensities of each of the two spectral channels are non-zero values that sums up to 1.0, and the relative intensities of the other channels are 0; and
4) for each basis set N_{ch}, where N_{ch} is the channel number, all of the labeled imager strands include a common type of label having emission in channel N_{ch}.

Item 41. The method of any of items 35-40, wherein the R values that represent the relative intensities of the more than one label is determined by:
(1) obtaining a calibration image of a sample which has been stained with just the multiple imager strands with two different types of labels alone and imaged under the same conditions as will be used for subsequent experiments;
(2) measuring the intensities from each spectral channel;
(3) dividing the image of one channel of the calibration image by the other to find a ratio image;
(4) creating a mask image that selects pixels in the calibration image wherever the intensity in the two channels is above a simple threshold (e.g. the threshold might be 20% of the maximum brightness for each);
(5) calculating the mean or median value, μ, of the ratio image at substantially all pixels in the mask image; and
(6) calculating the relative intensities, R₁ and R₂, in the two channels having the following relationships:

R₁/R₂=μ

R₁+R₂=1

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A multicolor multiplex imaging method comprising
(1) contacting a sample being tested for the presence of one or more targets with one or more target-specific binding partners, wherein each target-specific binding partner is linked to a nucleic acid strand and wherein target-specific binding partners of different specificity are linked to different nucleic acid strands;
(2) optionally removing unbound target-specific binding partners;
(3) contacting the sample with labeled imager strands, wherein in at least one occurrence of this step the labeled imager strands comprise (i) multiple labeled imager strands capable of binding the same nucleic acid strand associated with the target-specific binding partner, and to either the same or different domains within the nucleic acid strand, wherein the multiple imager strands comprise a different type of label and/or (ii) at least one labeled imager strand capable of binding the same nucleic acid strand associated with the target-specific binding partner, wherein the imager strand comprises more than one type of label;
(4) optionally removing unbound labeled imager strands;
(5) imaging the sample to detect bound labeled imager strands;
(6) optionally removing the bound labeled imager strands from the nucleic acid strands; and
(7) optionally repeating steps (1)-(6), or any subset thereof,
wherein imaging the sample to detect the bound labeled imager strands includes detecting Nₘ targets with N_{ch} labels used in the method wherein Nₘ is larger than N_{ch}, and optionally wherein Nₘ is an integer of 3, 4, 5, 6, 7, 8, 9, 10, 11, 12, 13, 14, or 15, and N_{ch} is an integer of 2, 3, 4, 5, or 6 smaller than N_{ch}.

2. The method of claim 1, wherein at least one of the labeled imager strand(s) of step (3)(i) and/or (ii) are capable of binding the same nucleic acid strand associated with the target-specific binding partner directly or indirectly.

3. The method of claim 1, wherein labeled imager strands of step (3)(i) and/or (ii) capable of binding the same nucleic acid strand associated with the target-specific binding partner bind to the same domain within the nucleic acid strand or a different domain within the nucleic acid strand.

4. The method of claim 1, wherein the method results in at least one target being labeled with at least two different types of labels.

5. The method of claim 1, wherein the multiple labeled imager strands capable of binding the same nucleic acid strand associated with the target-specific binding partner comprise an identical nucleotide sequence.

6. The method of claim 1, wherein the nucleic acid strand is a docking strand, and the multiple labeled imager strands comprise at least two labeled imager strands capable of binding the same docking strand, directly or indirectly, but comprising a different type of label, optionally wherein the multiple labeled imager strands comprise at least three labeled imager strands capable of binding the same docking strand, directly or indirectly, but comprising a different type of label, and/or optionally wherein the multiple labeled imager strands are capable of binding the same docking strand, but comprise a different type of label and are provided in equal amounts or unequal amounts.

7. The method of claim 6, wherein the multiple labeled imager strands comprise a first imager strand with a first label attached and a second imager strand with a second label attached, and both the first and second imager strands have a nucleotide sequence complementary to the same domain of the docking strand.

8. The method of claim 1, wherein the nucleic acid strand is a docking strand, and the docking strand has more than one domain of nucleotide sequence having complementarity to the labeled imager strand, optionally wherein the multiple labeled imager strands comprise a first imager strand with a first label attached, capable of binding to a first domain of the docking strand and a second imager strand with a second label attached, capable of binding to a second domain of the docking strand, the second domain having a nucleotide sequence different from the first domain.

9. The method of claim 1, wherein at least one of the at least one labeled imager strand comprises more than one type of label, optionally wherein the more than one type of label comprises at least two types of labels or at least three types of labels.

10. The method of claim 1, wherein the number of targets being detected, Nₘ, given the number of labels, N_{ch}, is represented by the following formula (1):
N_{ch}<Nₘ≤N_{ch}*(N_{ch}+1)/2 (1)
where Nₘ is an integer and N_{ch} is an integer chosen from 2, 3, 4, 5, and 6, and optionally wherein the number of targets being detected, Nₘ, given the number of labels, N_{ch}, is represented by the following formula (2):
Nₘ=N_{ch}*(N_{ch}+1)/2 (2)
where Nₘ is an integer and N_{ch} is an integer chosen from 2, 3, 4, 5, and 6.

11. The method of claim 1, wherein the imaging the sample to detect the labeled imager strands further comprises obtaining fluorescent spectral data from at least one image where each pixel contains the measured intensity in N_{ch} spectral channels for corresponding N_{ch} labels; and decoding the image to provide decoded images of the Nₘ targets.

12. The method of claim 11, wherein the decoding is conducted by processing the fluorescent spectral data with N_{ch} spectral channels pixel-by-pixel by performing the following:
(1) determining relative intensities in each spectral channel of a label or labels for each target;
(2) based on the determined relative intensities, grouping the targets into N_{ch}, mutually exclusive basis sets, the targets being the members of each basis set commonly having non-zero intensity in one of N_{ch} channels;
(3) given the relative spectral intensity of the members of each basis set, and given the measured intensity of the sample in each pixel in each channel, adjusting the levels of member of each basis set to produce the least error in matching the basis set with the measured intensities; and
(4) selecting the basis set with the least error and assigning each element of an output array with Nₘ values to the levels determined for the members of the selected set or to zero for members not of the selected set,
optionally wherein steps (3)-(4) are repeated for a portion of pixels of the input image or for all of pixels in the input image; and/or optionally wherein steps (3)-(4) are conducted in parallel for multiple pixels of the input image.

13. The method of claim 12, wherein the relative intensities in each spectral channel for a label or labels in step (1) are determined by capturing an image of a calibration sample containing multiple labeled imager strands for the same target, and obtaining the relative intensities in spectral channels; repeating the same for the multiple labeled imager strands associated with different targets.

14. The method of claim 12, wherein the relative intensities in each spectral channel of a label or labels for each target in step (1) are determined to meet the following:
1) for each target, the sum of the relative intensities in each N_{ch} spectral channels is 1;
2) when the target is associated with only one type of label N_{ch}, the relative intensity in corresponding spectral channel N_{ch} is 1.0 and the relative intensities of the other channels are 0;
3) when the target is associated with more than one type of label, the relative intensities of each of the two spectral channels are non-zero values that sums up to 1.0, and the relative intensities of the other channels are 0; and
4) for each basis set N_{ch}, where N_{ch} is the channel number, all of the labeled imager strands include a common type of label having emission in channel N_{ch}.

15. The method of claim 12, wherein the R values that represent the relative intensities of the more than one label is determined by:
(1) obtaining a calibration image of a sample which has been stained with just the multiple imager strands with two different types of labels alone and imaged under the same conditions as will be used for subsequent experiments;
(2) measuring the intensities from each spectral channel;
(3) dividing the image of one channel of the calibration image by the other to find a ratio image;
(4) creating a mask image that selects pixels in the calibration image wherever the intensity in the two channels is above a simple threshold (e.g. the threshold might be 20% of the maximum brightness for each);
(5) calculating the mean or median value, μ, of the ratio image at substantially all pixels in the mask image; and
(6) calculating the relative intensities, R₁ and R₂, in the two channels having the following relationships:
R₁/R₂=μ
R₁+R₂=1

16. A kit for detecting Nₘ targets with N_{ch} labels provided in the kit wherein Nₘ is larger than N_{ch}, comprising:
1) target-specific binding partners linked to nucleic acid strands, wherein target-specific binding partners of different specificity are linked to different nucleic acid strands;
2) labeled imager strands comprising (i) multiple labeled imager strands capable of binding the same nucleic acid strand associated with the target-specific binding partner, to either the same or different domains within the nucleic acid strand, wherein the multiple imager strands comprise a different type of label and/or (ii) at least one labeled imager strand capable of binding the same nucleic acid strand associated with the target-specific binding partner, wherein the imager strand comprises more than one type of label; and
3) optional buffers, amplification reagents, and/or reagents to remove bound imager strands.

17. A system for detecting a plurality of targets from fluorescence spectral data, wherein the number of targets to be detected, Nₘ, given the number of labels, N_{ch}, is represented by the following formula:
N_{ch}<Nₘ≤N_{ch}*(N_{ch}+1)/2,
where Nₘ and N_{ch} are an integer, the system comprising a fluorescent microscope, a light source, a detector, a computer processor operably connected with the detector;
and a tangible non-transitory storage medium having computer-readable instructions embedded therein which, when loaded onto the computer processor, cause the processor to conduct the following:
(1) determining relative intensities in each spectral channel of a label or labels for each target;
(2) based on the determined relative intensities, grouping the targets into N_{ch}, mutually exclusive basis sets, the targets being the members of each basis set commonly having non-zero intensity in one of N_{ch} channels;
(3) given the relative spectral intensity of the members of each basis set, and given the measured intensity of the sample in each pixel in each channel, adjusting the levels of member of each basis set to produce the least error in matching the basis set with the measured intensities; and
(4) selecting the basis set with the least error and assigning each element of an output array with Nₘ values to the levels determined for the members of the selected set or to zero for members not of the selected set,
optionally wherein steps (3)-(4) are repeated for a portion of pixels of the input image or for all of pixels in the input image; and/or optionally wherein steps (3)-(4) are conducted in parallel for multiple pixels of the input image.

18. The method of claim 17, wherein the relative intensities in each spectral channel for a label or labels in step (1) are determined by capturing an image of a calibration sample containing multiple labeled imager strands for the same target, and obtaining the relative intensities in spectral channels; repeating the same for the multiple labeled imager strands associated with different targets.

19. The method of claim 17, wherein the relative intensities in each spectral channel of a label or labels for each target in step (1) are determined to meet the following:
1) for each target, the sum of the relative intensities in each N_{ch} spectral channels is 1;
2) when the target is associated with only one type of label N_{ch}, the relative intensity in corresponding spectral channel N_{ch} is 1.0 and the relative intensities of the other channels are 0;
3) when the target is associated with more than one type of label, the relative intensities of each of the two spectral channels are non-zero values that sums up to 1.0, and the relative intensities of the other channels are 0; and
4) for each basis set N_{ch}, where N_{ch} is the channel number, all of the labeled imager strands include a common type of label having emission in channel N_{ch}.

20. The method of claim 17, wherein the R values that represent the relative intensities of the more than one label is determined by:
(1) obtaining a calibration image of a sample which has been stained with just the multiple imager strands with two different types of labels alone and imaged under the same conditions as will be used for subsequent experiments;
(2) measuring the intensities from each spectral channel;
(3) dividing the image of one channel of the calibration image by the other to find a ratio image;
(4) creating a mask image that selects pixels in the calibration image wherever the intensity in the two channels is above a simple threshold (e.g. the threshold might be 20% of the maximum brightness for each);
(5) calculating the mean or median value, μ, of the ratio image at every pixel in the mask image; and
(6) calculating the relative intensities, R₁ and R₂, in the two channels having the following relationships:
R₁/R₂=μ
R₁+R₂=1
